SEQUENCE LISTING (43 sequences)

SEQ ID NO: 1 - protein, 130 aa, Homo sapiens
LCQGSNYSTCASCPSCPDRWMKYGNHCYYFSVEEKDWNSSLEFCLARDSHLLVITDNQEMSLLQVFLSEAFCWIGLRNNSGWRWEDGSPLNFSRISSNSFVQTCGAINKNGLQASSCEVPLHWVCKKVRL

SEQ ID NO: 2 - protein, 130 aa, Macaca fascicularis
LCQGSKYSTCASCPSCPDHWMKYGNHCYYFSVEKKDWISSLEFCLARDSHLLMITDKQEMSLLQDFLSEAFHWVGLRNNSGWRWEDGSPLNFSRIYSNSLVQTCGAINKNSLQASSCEVSLQWVCKKVSP

SEQ ID NO: 3 - protein, 728 aa, Homo sapiens
DWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 4 - protein, 125 aa, Artificial Sequence
  Description of Artificial Sequence Synthetic polypeptide
QVTLRESGPALVKPTQTLTLTCTVSGFSLSTFGMGVGWIRQPPGKALEWLAHIWWDDDKWYELALKSRLTISKDTSKNQVVLTITNMDPVDTATYYCARVIYYGSRSAYYSMDYWGQGTTVTVSS

SEQ ID NO: 5 - protein, 115 aa, Artificial Sequence
  Description of Artificial Sequence Synthetic polypeptide
DVVMTQTPLSLSVTPGQPASISCKSSQSIVHSNAHTYLEWYLQKPGQSPQLLIYKVSNRFSGVPDRFSGSGSGTDFTLKISRVEAEDVGVYYCFQGSHVPVTFGQGTKLEIKRTV

SEQ ID NO: 6 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 104 aa, Artificial Sequence
  Description of Artificial Sequence Synthetic polypeptide
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 455 aa, Artificial Sequence
  Description of Artificial Sequence Synthetic polypeptide
QVTLRESGPALVKPTQTLTLTCTVSGFSLSTFGMGVGWIRQPPGKALEWLAHIWWDDDKWYELALKSRLTISKDTSKNQVVLTITNMDPVDTATYYCARVIYYGSRSAYYSMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 219 aa, Artificial Sequence
  Description of Artificial Sequence Synthetic polypeptide
DVVMTQTPLSLSVTPGQPASISCKSSQSIVHSNAHTYLEWYLQKPGQSPQLLIYKVSNRFSGVPDRFSGSGSGTDFTLKISRVEAEDVGVYYCFQGSHVPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 9 aa, Artificial Sequence
  Description of Artificial Sequence Synthetic peptide
GFSLSTFGM

SEQ ID NO: 11 - protein, 5 aa, Artificial Sequence
  Description of Artificial Sequence Synthetic peptide
WWDDD

SEQ ID NO: 12 - protein, 15 aa, Artificial Sequence
  Description of Artificial Sequence Synthetic peptide
VIYYGSRSAYYSMDY

SEQ ID NO: 13 - protein, 16 aa, Artificial Sequence
  Description of Artificial Sequence Synthetic peptide
KSSQSIVHSNAHTYLE

SEQ ID NO: 14 - protein, 7 aa, Artificial Sequence
  Description of Artificial Sequence Synthetic peptide
KVSNRFS

SEQ ID NO: 15 - protein, 9 aa, Artificial Sequence
  Description of Artificial Sequence Synthetic peptide
FQGSHVPVT

SEQ ID NO: 16 - protein, 12 aa, Artificial Sequence
  Description of Artificial Sequence Synthetic peptide
GFSLSTFGMGVG

SEQ ID NO: 17 - protein, 9 aa, Artificial Sequence
  Description of Artificial Sequence Synthetic peptide
HIWWDDDKW

SEQ ID NO: 18 - protein, 15 aa, Artificial Sequence
  Description of Artificial Sequence Synthetic peptide
VIYYGSRSAYYSMDY

SEQ ID NO: 19 - protein, 16 aa, Artificial Sequence
  Description of Artificial Sequence Synthetic peptide
KSSQSIVHSNAHTYLE

SEQ ID NO: 20 - protein, 7 aa, Artificial Sequence
  Description of Artificial Sequence Synthetic peptide
KVSNRFS

SEQ ID NO: 21 - protein, 9 aa, Artificial Sequence
  Description of Artificial Sequence Synthetic peptide
FQGSHVPVT

SEQ ID NO: 22 - protein, 7 aa, Artificial Sequence
  Description of Artificial Sequence Synthetic peptide
TFGMGVG

SEQ ID NO: 23 - protein, 16 aa, Artificial Sequence
  Description of Artificial Sequence Synthetic peptide
HIWWDDDKWYELALKS

SEQ ID NO: 24 - protein, 15 aa, Artificial Sequence
  Description of Artificial Sequence Synthetic peptide
VIYYGSRSAYYSMDY

SEQ ID NO: 25 - protein, 16 aa, Artificial Sequence
  Description of Artificial Sequence Synthetic peptide
KSSQSIVHSNAHTYLE

SEQ ID NO: 26 - protein, 7 aa, Artificial Sequence
  Description of Artificial Sequence Synthetic peptide
KVSNRFS

SEQ ID NO: 27 - protein, 9 aa, Artificial Sequence
  Description of Artificial Sequence Synthetic peptide
FQGSHVPVT

SEQ ID NO: 28 - protein, 8 aa, Artificial Sequence
  Description of Artificial Sequence Synthetic peptide
STFGMGVG

SEQ ID NO: 29 - protein, 12 aa, Artificial Sequence
  Description of Artificial Sequence Synthetic peptide
WLAHIWWDDDKW

SEQ ID NO: 30 - protein, 16 aa, Artificial Sequence
  Description of Artificial Sequence Synthetic peptide
ARVIYYGSRSAYYSMD

SEQ ID NO: 31 - protein, 12 aa, Artificial Sequence
  Description of Artificial Sequence Synthetic peptide
VHSNAHTYLEWY

SEQ ID NO: 32 - protein, 10 aa, Artificial Sequence
  Description of Artificial Sequence Synthetic peptide
LLIYKVSNRF

SEQ ID NO: 33 - protein, 8 aa, Artificial Sequence
  Description of Artificial Sequence Synthetic peptide
FQGSHVPV

SEQ ID NO: 34 - protein, 10 aa, Artificial Sequence
  Description of Artificial Sequence Synthetic peptide
GFSLSTFGMG

SEQ ID NO: 35 - protein, 7 aa, Artificial Sequence
  Description of Artificial Sequence Synthetic peptide
IWWDDDK

SEQ ID NO: 36 - protein, 17 aa, Artificial Sequence
  Description of Artificial Sequence Synthetic peptide
ARVIYYGSRSAYYSMDY

SEQ ID NO: 37 - protein, 11 aa, Artificial Sequence
  Description of Artificial Sequence Synthetic peptide
QSIVHSNAHTY

SEQ ID NO: 38 - protein, 2 aa, Artificial Sequence
  Description of Artificial Sequence Synthetic peptide
KV

SEQ ID NO: 39 - protein, 9 aa, Artificial Sequence
  Description of Artificial Sequence Synthetic peptide
FQGSHVPVT

SEQ ID NO: 40 - DNA, 1428 bp, Artificial Sequence
  Description of Artificial Sequence Synthetic polynucleotide
atgggctggtcctgcatcattctgtttctggtggctaccgccaccggcgctcattctcaagtgacactgagagagtctggccccgctctggtcaagcctacacagaccctgacactgacctgcaccgtgtctggcttctccctgtctacctttggcatgggcgtcggctggattagacagcctcctggaaaagccctggaatggctggcccacatttggtgggacgacgacaagtggtacgagctggctctgaagtcccggctgaccatctccaaggacacctccaagaatcaggtggtgctgacaatcaccaacatggaccctgtggacaccgccacctactactgcgccagagtgatctactacggctccagatccgcctactactccatggattattggggccagggcaccaccgtgaccgtgtcctctgcttctacaaagggcccctctgtgttccctctggctccttcctctaaatccacctctggcggaaccgctgctctgggctgtctcgtgaaggattacttccctgagcctgtgacagtgtcctggaatagcggtgctctgacatccggcgtgcacacctttccagctgtgctgcagtcctctggcctgtactctctgtcctctgtcgtgacagtgccttccagctctctgggcacccagacctacatctgcaacgtgaaccacaagccttccaacaccaaggtggacaagaaggtggaacccaagtcctgcgacaagacccacacctgtcctccatgtcctgctccagaagctgctggcgctccctccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatcagcaaggctaagggccagcctcgggaacctcaggtttacaccctgcctccatctcgggaagagatgacaaagaaccaggtgtccctgacctgcctggtcaagggcttctacccttccgacattgccgtggaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacccagaagtccctgtctctgtcccctggcaagtgatga

SEQ ID NO: 41 - DNA, 720 bp, Artificial Sequence
  Description of Artificial Sequence Synthetic polynucleotide
atgggctggtcctgcatcattctgtttctggtggctaccgccaccggcgctcattctgatgtggtcatgacacagacccctctgagcctgtctgtgacacctggacagcctgcctccatctcctgcaagtcctctcagtccatcgtgcacagcaacgcccacacctacctggaatggtatctgcagaagcccggccagtctcctcagctgctgatctacaaggtgtccaacagattctctggcgtgcccgacagattcagcggctctggctctggcaccgacttcaccctgaagatctctagagtggaagccgaggacgtgggcgtgtactactgtttccaaggctctcacgtgcccgtgacctttggccagggaacaaagctggaaatcaagcggaccgtggccgctccttccgtgttcatctttccaccttccgacgagcagctgaagtccggcacagcttctgtcgtgtgcctgctgaacaacttctaccctcgggaagccaaggtgcagtggaaagtggataatgccctgcagtccggcaactcccaagagtctgtgaccgagcaggactccaaggactctacctactctctgtcctccacactgaccctgtccaaggccgactacgagaagcacaaggtgtacgcctgtgaagtgacccaccagggactgtctagccccgtgaccaagtctttcaaccggggcgagtgctgatga

SEQ ID NO: 42 - protein, 15 aa, Artificial Sequence
  Description of Artificial Sequence Synthetic peptide
GGGGSGGGGSGGGGS

SEQ ID NO: 43 - protein, 5 aa, Artificial Sequence
  Description of Artificial Sequence Synthetic peptide
CNGRC

(19) United States
(12) Patent Application Publication
(10) Pub. No.: US 2024/0174754 A1
(43) Pub. Date: May 30, 2024

(54) ANTI-KLRG1 ANTIBODIES

(71) Applicant: ABCURO, INC., Newton, MA (US)

(72) Inventors: Stefano V. GULLA, Newton, MA (US); Kenneth Evan THOMPSON, Newton, MA (US)

(21) Appl. No.: 18/552,515

(22) PCT Filed: Mar. 25, 2022
(86) PCT No.: PCT/US2022/021945

§ 371 Date: Sep. 26, 2023

Related U.S. Application Data
(60) Provisional application No. 63/166,663, filed on Mar. 26, 2021.
(60) Provisional application No. 63/294,436, filed on Dec. 29, 2021.

(51) Int. Cl. C07K 16/28; A61K 45/06

(52) U.S. Cl. CPC C07K 16/2851; A61K 45/06; C07K 2317/31; C07K 2317/54; C07K 2317/55; C07K 2317/94

(57) ABSTRACT

The disclosure relates to antibodies, or antigen-binding fragments thereof, that specifically bind to killer cell lectin-like receptor G1 (KLRG1). Such antibodies, or antigen-binding fragments thereof, are useful for various therapeutic or diagnostic purposes, including treatment of cancers and to increase the effectiveness of vaccines.

### CROSS REFERENCE TO RELATED APPLICATIONS

This application claims the benefit of U.S. Provisional Application No. 63/166,663, filed on Mar. 26, 2021 and U.S. Provisional Application No. 63/294,436, filed on Dec. 29, 2021, the contents of which are each incorporated herein by reference in their entireties.

### TECHNICAL FIELD

The technical field relates to inhibition of the lymphocyte co-inhibitory receptor Killer Cell Lectin-Like Receptor Subfamily G Member 1.

### REFERENCE TO A SEQUENCE LISTING

This application contains a Sequence Listing in computer readable form. The computer readable form is incorporated herein by reference. Said ASCII copy, created on Mar. 1, 2022, is named 770808_000043_SL.txt and is 32,100 bytes in size.

### BACKGROUND

Lymphocyte co-inhibitory receptors modulate the action of the adaptive immune system (see e.g. WO2020/060781). The action of co-inhibitory receptors is generally carried out by binding of a ligand to the extracellular domain of the co-inhibitory receptor followed by recruitment of intracellular phosphatases by an immunoreceptor tyrosine-based inhibition motif (ITIM) located in the intracellular domain of the co-inhibitory receptor. The action of co-inhibitory receptors is generally to dampen the immune response of T-cell receptor (TCR) engagement. In recent years it has been shown that agents that block the activity of co-inhibitory receptors can be used to as efficacious treatments for cancer and infectious diseases.

### SUMMARY OF THE INVENTION

In one aspect, the present disclosure relates to an antibody, or antigen-binding fragment thereof, that specifically binds to an extracellular domain of KLRG1 which comprises a heavy chain variable region comprising SEQ ID NO: 4. The antibody, or antigen-binding fragment thereof may further comprise a light chain comprising a CDR-L1 selected from the group consisting of SEQ ID NO: 13, SEQ ID NO: 19, SEQ ID NO:25, SEQ ID NO:31, and SEQ ID NO:37; a CDR-L2 selected from the group consisting of SEQ ID NO: 14, SEQ ID NO:20, SEQ ID NO:26, SEQ ID NO:32, and SEQ ID NO:38; and a CDR-L3 selected from the group consisting of SEQ ID NO: 15, SEQ ID NO:21, SEQ ID NO:27, SEQ ID NO:33, and SEQ ID NO:39. The CDR-L1, CDR-L2, and CDR-L3 may be SEQ ID NO: 13, SEQ ID NO: 14 and SEQ ID NO: 15, respectively; SEQ ID NO:19, SEQ ID NO:20 and SEQ ID NO:21, respectively; SEQ ID NO:25, SEQ ID NO:26 and SEQ ID NO:27, respectively; SEQ ID NO:31, SEQ ID NO:32 and SEQ ID NO:33, respectively; or SEQ ID NO:37, SEQ ID NO:38 and SEQ ID NO:39, respectively.

In another aspect, the disclosure relates to an antibody, or antigen-binding fragment thereof, that specifically binds to an extracellular domain of KLRG1, which comprises a light chain variable region comprising SEQ ID NO: 5. The antibody, or antigen-binding fragment thereof, of claim 4, may further comprise a heavy chain comprising a CDR-L1 selected from the group consisting of SEQ ID NO: 10, SEQ ID NO: 16, SEQ ID NO:22, SEQ ID NO:28, SEQ ID NO:34; a CDR-L2 selected from the group consisting of SEQ ID NO:11, SEQ ID NO: 17, SEQ ID NO:23, SEQ ID NO:29, SEQ ID NO:35; and a CDR-L3 selected from the group consisting of SEQ ID NO: 12, SEQ ID NO: 18, SEQ ID NO:24, SEQ ID NO:30, SEQ ID NO:36. The CDR-H1, CDR-H2, and CDR-H3 may be SEQ ID NO: 10, SEQ ID NO:11 and SEQ ID NO: 12, respectively; SEQ ID NO: 16, SEQ ID NO: 17 and SEQ ID NO: 18, respectively; SEQ ID NO:22, SEQ ID NO:23 and SEQ ID NO:24, respectively; SEQ ID NO:28, SEQ ID NO:29 and SEQ ID NO:30, respectively; or SEQ ID NO:34, SEQ ID NO:35 and SEQ ID NO:36, respectively.

In one aspect, the disclosure relates to an antibody, or antigen-binding fragment thereof, that specifically binds to an extracellular domain of KLRG1 which comprises: (a) a heavy chain variable region comprising SEQ ID NO: 4, and (b) a light chain variable region comprising SEQ ID NO: 5. The antibody, or antigen-binding fragment thereof, may comprise a heavy chain comprising SEQ ID NO: 8. The antibody, or antigen-binding fragment thereof may comprise a light chain comprising SEQ ID NO: 9. The antibody, or antigen-binding fragment thereof may comprise (a) a heavy chain comprising SEQ ID NO: 8, and (b) a light chain comprising SEQ ID NO: 9.

The antigen-binding fragment may be a F(ab) fragment. The antigen-binding fragment may be a F(ab₂) fragment. The antibody or antigen-binding fragment thereof may be bispecific.

In one aspect, the disclosure relates to methods of treating cancer, comprising administering to a cancer patient the antibody or antigen-binding fragments disclosed herein. The method may also include administering a checkpoint inhibitor to the cancer patient. The checkpoint inhibitor may be administered at the same time as administering the antibody or antigen-binding fragment thereof. The checkpoint inhibitor may be administered within one day, one week, or one month of the antibody or antigen-binding fragment thereof. The cancer may be one or more of breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma

The disclosure also relates to an adjunct therapy, comprising administering to a subject who is undergoing treatment with a checkpoint inhibitor an antibody or antigen-binding fragment thereof disclosed herein, thereby to perform said adjunct therapy.

### BRIEF DESCRIPTION OF THE DRAWINGS

This disclosure will be more fully understood from the following detailed description taken in conjunction with the accompanying drawings.

FIG. 1 is a graph showing PA015 binding to CHOK1 cells expressing human KLRG1.

FIG. 2 is a graph showing PA015 inhibition of E-cadherin binding to CHOK1 cells expressing KLRG1 in a relative binding assay.

FIG. 3 is a graph showing PA015 inhibition of E-cadherin binding to CHOK1 cells expressing KLRG1, compared with HG1N01 and HG1N02, in a relative binding assay.

FIG. 4 shows graphs of PA015 binding to human CD8+ T cells from two donors, compared with HG1N02, in a FACS binding assay.

FIG. 5 is a graph showing serum concentration of PA015 over time in a cynomolgus monkey pharmacokinetic assay described in Example 9.

FIG. 6 is a graph showing serum concentration of HG1N01 over time in the cynomolgus monkey pharmacokinetic assay described in Example 9.

FIG. 7 shows a graph of deamidation at particular light chain (LC) and heavy chain (HC) residues of PA015, compared with HG1N02.

### DETAILED DESCRIPTION

Certain exemplary embodiments are described to provide an overall understanding of the principles of the structure, function, manufacture, and use of the antibodies, fragments thereof, compositions of matter, kits, and related methods and therapies disclosed herein.

Killer cell lectin-like receptor G1 (KLRG1) is a type II transmembrane protein which can function as a co-inhibitory receptor by modulating the activity of T and NK cells. The extracellular portion of KLRG1 contains a C-type lectin domain whose known ligands are cadherins. The intracellular portion of KLRG1 contains an immunoreceptor tyrosine-based inhibitory motif (ITIM) domain responsible for co-inhibition of T cell receptor (TCR) mediated signaling. KLRG1 ligands include E-cadherin, N-cadherin, R-cadherin, and combinations thereof.

In humans, KLRG1 expression is generally confined to cells of the immune system, and specifically to CD8 positive T cells, NK cells, and, to a lesser extent, CD4 positive T cells. KLRG1 expression has been associated with the late differentiated phenotype. As antigen specific T cells differentiate, they can acquire increased expression of cytotoxic molecules, and therefore can have increased cytotoxic potential. The biological function of KLRG1 is to inhibit cytotoxicity and proliferation of these T cells under certain circumstances. In murine cancer models, the blockade of KLRG1's interaction with its ligands has been shown to be beneficial to controlling tumor growth or metastases.

Unless otherwise defined, all technical and scientific terms used herein have the same meaning as commonly understood by one skilled in the art to which this present disclosure belongs. The terminology used in the description of the present disclosure herein is for the purpose of describing particular embodiments only and is not intended to be limiting of the present disclosure and any invention(s) described or otherwise provided for herein.

Amino acids are represented herein by either the one-letter code, or the three-letter code, both in accordance with 37 C.F.R. § 1.822 and established usage.

All publications, patent applications, patents, patent publications, and other references cited herein are incorporated by reference in their entireties for the teachings relevant to the sentence and/or paragraph in which the reference is presented.

### Definitions

As used in the description of the present disclosure and the appended claims, the singular forms “a,” “an,” and “the” are intended to include the plural forms as well, unless the context clearly indicates otherwise.

As used herein, “and or” refers to and encompasses any and all possible combinations of one or more of the associated listed items, as well as the lack of combinations when interpreted in the alternative (“or”).

The terms “about” and “approximately” as used herein when referring to a measurable value such as an amount of polypeptide, dose, time, temperature, enzymatic activity or other biological activity and the like, is meant to encompass variations of ±20%, ±10%, ±5%, ±1%, ±0.5%, or even ±0.1% of the specified amount.

The transitional phrase “consisting essentially of” means that the scope of a claim is to be interpreted to encompass the specified materials or steps recited in the claim, “and those that do not materially affect the basic and novel characteristic(s)” of the claimed invention. See In re Herz, 537 F.2d 549, 551-52, 190 USPQ 461, 463 (CCPA 1976) (emphasis in the original); see also MPEP § 2111.03.

The term “consists essentially of” (and grammatical variants), as applied to a polynucleotide or polypeptide sequence of this present disclosure, means a polynucleotide or polypeptide that consists of both the recited sequence (e.g., SEQ ID NO) and a total of ten or less (e.g., 1, 2, 3, 4, 5, 6, 7, 8, 9, or 10) additional amino acids on the N-terminal and/or C-terminal ends of the recited sequence such that the function of polypeptide is not materially altered. The total of ten or less additional amino acids can include the total number of additional amino acids on both ends added together.

An “effective amount” as used herein is an amount that provides a desired effect. The term “effective amount” refers to a dosage or amount that is sufficient to reduce the activity of KLRG1 to result in amelioration of symptoms in a patient or to achieve a desired biological outcome, e.g., reduced activity of KLRG1, modulation of lymphocyte co-inhibition response, increased or decreased activation of cytotoxic T and NK cells, or increased or decreased release of IFNγ by cytotoxic T cells or NK cells.

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject.

By the terms “treat,” “treating,” or “treatment of,” it is intended that the severity of the condition of the subject is reduced, or at least partially improved or modified, and that some alleviation, mitigation, or decrease in at least one clinical symptom is achieved.

The term “cancer” as used herein refers to any benign or malignant abnormal growth of cells. Examples include, without limitation, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is selected from the group of tumor-forming cancers. A person skilled in the art will recognize which cancers fall within the purview of that group.

The term “isolated” can refer to a polypeptide that is substantially free of cellular material, viral material, and/or culture medium (when produced by recombinant DNA techniques), or chemical precursors or other chemicals (when chemically synthesized). Moreover, an “isolated fragment” is a fragment of a polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose. Thus, the term “isolated” refers to a molecule that is substantially free of its natural environment. For instance, an isolated protein is substantially free of cellular material or other proteins from the cell or tissue source from which it is derived. The term “isolated” also refers to preparations where the isolated protein is sufficiently pure to be administered as a pharmaceutical composition, or approximately at least 70-80% (w/w) pure, more preferably, approximately at least 80-90% (w/w) pure, even more preferably, approximately 90-95% pure; and, most preferably, at least 95%, at least 96%, at least 97%, at least 98%, at least 99%, or at least 100% (w/w) pure.

The term “fragment” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or nearly identical (e.g., approximately 90%, approximately 92%, approximately 95%, approximately 98%, approximately 99% identical) to the reference polypeptide or amino acid sequence. Such a polypeptide fragment according to the present disclosure may be, where appropriate, included in a larger polypeptide of which it is a constituent. In some embodiments, such fragments can comprise, consist essentially of, and/or consist of peptides having a length of at least about 4, about 6, about 8, about 10, about 12, about 15, about 20, about 25, about 30, about 35, about 40, about 45, about 50, about 75, about 100, about 150, about 200, or more consecutive amino acids of a polypeptide or amino acid sequence according to the present disclosure.

As used herein, the terms “protein” and “polypeptide” are used interchangeably and encompass both peptides and proteins, unless indicated otherwise.

The term “antibody” or “antibodies” as used herein refers to all types of immunoglobulins, including IgG, IgM, IgA, IgD, and IgE. The antibody can be monoclonal or polyclonal and can be of any species of origin, including, for example, mouse, rat, rabbit, horse, goat, sheep, camel, or human, or can be a chimeric antibody. The antibodies can be recombinant monoclonal antibodies produced according to the methods disclosed, for example, in U.S. Pat. No. 4,474,893 or U.S. Pat. No. 4,816,567. The antibodies can also be chemically constructed, for example, according to the methods disclosed in U.S. Pat. No. 4,676,980.

The terms “antigen-binding domain,” “antigen-binding fragment,” and “binding fragment” refer to a part of an antibody molecule that comprises amino acids responsible for the specific binding between the antibody and the antigen. In instances where an antigen is large, the antigen-binding domain may only bind to a part of the antigen. A portion of the antigen molecule that is responsible for specific interactions with the antigen-binding domain is referred to as “epitope” or “antigenic determinant.”

Though an antigen-binding domain typically comprises an antibody light chain variable region (VL) and an antibody heavy chain variable region (VH), it does not necessarily have to comprise both. For example, a Fd antibody fragment consists only of a VH domain, but still retains some antigen-binding function of the intact antibody.

Anti-KLRG1 antibodies comprise antibody constant regions or parts thereof. For example, a VL domain may have attached, at its C terminus, antibody light chain constant domains including human Cκ or Cλ chains. Similarly, a specific antigen-binding domain based on a VH domain may have attached all or part of an immunoglobulin heavy chain derived from any antibody isotope, e.g., IgG, IgA, IgE, and IgM and any of the isotope sub-classes, which include but are not limited to, IgG1 and IgG4. The DNA and amino acid sequences for the C-terminal fragment of are well known in the art.

The terms “specific interaction” and “specific binding” refer to two molecules forming a complex that is relatively stable under physiologic conditions. Specific binding can be characterized by a high affinity and a low to moderate capacity, as distinguished from non-specific binding, which usually has a low affinity with a moderate to high capacity. Typically, binding is considered specific when the affinity constant KA is higher than approximately 10⁶ M⁻¹, or more preferably higher than approximately 10⁸ M⁻¹. If necessary, non-specific binding can be reduced without substantially affecting specific binding, for example, by varying the binding conditions. The appropriate binding conditions such as concentration of antibodies, ionic strength of the solution, temperature, time allowed for binding, concentration of a blocking agent (e.g., serum albumin, milk casein), etc., may be optimized by a skilled artisan using routine techniques.

In certain embodiments, the antibodies can specifically bind an epitope within the extracellular domain (ECD) of human or monkey KLRG1, with an affinity, as expressed in KD, of at least about 2 nM, about 1 nm, about 100 pM, about 10 pM, or about 5 pM. The amino acid sequences of ECDs of human and cynomolgus KLRG1 are set out in SEQ ID NO:1 and SEQ ID NO:2, and the amino acid sequence of human E-Cadherin is set forth in SEQ ID NO: 3, below.

### Antibodies and Compositions

The present disclosure describes antibodies and fragments thereof that specifically bind to the extracellular domain of KLGR1 of cells by interfering with the binding of E-cadherin, R-cadherin, and/or N-cadherin to the extracellular domain of KLRG1. By interfering with binding of an E-cadherin, R-cadherin, and/or N-cadherin ligand to KLRG1, such antibodies prolong the cytotoxicity of T- and NK-cells which bear KLRG1 on their surface. The antibodies described herein can be used as effective therapeutic agents for treatment of cancer either as a monotherapy, or in combination with other immunotherapy agents (such as checkpoint inhibitors, e.g., anti-PD-1 antibodies, anti-PD-L1 antibodies, or anti-CTLA4 antibodies), or in combination with chemotherapy agents, or cancer vaccines. Such antibodies, or fragments thereof, can be used as an adjunct therapy for cancer treatment, regardless of whether the cancer cells express KLRG1.

The subunit structures and three-dimensional configurations of different classes of immunoglobulins are well known in the art. Briefly, each light chain can be composed of an N-terminal variable domain (VL) and a constant domain (CL). Each heavy chain can be composed of an N-terminal variable domain (VH), three or four constant domains (CH), and a hinge region. The CH domain most proximal to VH is designated as CH1. The VH and VL domains consist or comprise of four regions of relatively conserved sequence called framework regions (FR1, FR2, FR3, and FR4), which form a scaffold for three regions of hypervariable sequence called complementarity determining regions (CDRs). The CDRs can contain most of the residues responsible for specific interactions with the antigen. The three CDRs are referred to as CDR1, CDR2, and CDR3. CDR constituents on the heavy chain are referred to as H1, H2, and H3, while CDR constituents on the light chain are referred to as L1, L2, and L3, accordingly. CDR3 and particularly H3, are the greatest source of molecular diversity within the antigen-binding domain. H3, for example, can be as short as two amino acid residues or greater than 26.

The Fab fragment (Fragment antigen-binding), or F(ab), consists of or comprises the VH-CH1 and VL-CL domains covalently linked by a disulfide bond between the constant regions. Known to those skilled in the art, a Fab (50,000 daltons) is a monovalent fragment that is produced from IgG and IgM, consisting or comprising of the VH, CH1 and VL, CL regions, linked by an intramolecular disulfide bond. To overcome the tendency of non-covalently linked VH and VL domains in the Fv to dissociate when co-expressed in a host cell, a so-called single chain (sc) Fv fragment (scFv) can be constructed. In a scFv, a flexible and adequately long polypeptide links either the C-terminus of the VH to the N-terminus of the VL, or the C-terminus of the VL to the N-terminus of the VH. Most commonly, a 15-residue (Gly4Ser)3 peptide (SEQ ID NO: 42) can be used as a linker, but other linkers are also known in the art.

Antibody diversity is a result of combinatorial assembly of multiple germline genes encoding variable regions and a variety of somatic events. The somatic events can include recombination of variable gene segments with diversity (D) and joining (J) gene segments to make a complete VH region and the recombination of variable and joining gene segments to make a complete VL region. The recombination process itself is imprecise, resulting in the loss or addition of amino acids at the V(D)J junctions. These mechanisms of diversity occur in the developing B cell prior to antigen exposure. After antigenic stimulation, the expressed antibody genes in B cells can undergo somatic mutation.

Based on the estimated number of germline gene segments, the random recombination of these segments, and random VH-VL pairing, up to approximately 1.6×10⁷ different antibodies can be produced according to Fundamental Immunology, 3^{rd} ed., ed. Paul, Raven Press, New York, N.Y., 1993. When other processes which contribute to antibody diversity (such as somatic mutation) are taken into account, it is thought that upwards of approximately 1×10¹⁰ different antibodies could be potentially generated, as supported by Immunoglobulin Genes, 2^{nd} ed., eds. Jonio et al., Academic Press, San Diego, Calif., 1995. Because of the many processes involved in antibody diversity, it is highly unlikely that independently generated antibodies will have identical amino acid sequences in the CDRs.

The present disclosure provides novel antibody variable heavy and light chain regions (VH and VL) and antibody heavy and light chains derived from human immunoglobulin gene libraries, which are effective in blocking KLRG1 signaling in cells expressing cell surface KLGR1. These heavy and light chains and their variable regions provide a scaffold structure for carrying CDRs. A CDR can generally be, though is not limited to, an antibody heavy or light chain, or a portion thereof, in which the CDR is located at a location corresponding to the CDR of naturally occurring VH and VL. The structures and locations of immunoglobulin variable domains may be determined, for example, as described in Kabat et al., Sequences of Proteins of Immunological Interest, No. 91-3242, National Institutes of Health Publications, Bethesda, Md., 1991.

TABLE 1
Amino acid sequences of human and cynomolgus KLRG1 ECD and human E-

| SEQ ID | | |
|---|---|---|
| NO: | PROTEIN | AMINO ACID SEQUENCE |
| SEQ ID | Human | LCQGSNYSTCASCPSCPDRWMKYGNHCYYF |
| NO: 1 | KLRG1 ECD | SVEEKDWNSSLEFCLARDSHLLVITDNQEMS |
| | | LLQVFLSEAFCWIGLRNNSGWRWEDGSPLN |
| | | FSRISSNSFVQTCGAINKNGLQASSCEVPLH |
| | | WVCKKVRL |
| SEQ ID | Cynomolgus | LCQGSKYSTCASCPSCPDHWMKYGNHCYY |
| NO: 2 | KLRG1 ECD | FSVEKKDWISSLEFCLARDSHLLMITDKQEM |
| | | SLLQDFLSEAFHWVGLRNNSGWRWEDGSPL |
| | | NFSRIYSNSLVQTCGAINKNSLQASSCEVSLQ |
| | | WVCKKVSP |
| SEQ ID | Human E- | DWVIPPISCPENEKGPFPKNLVQIKSNKDKEG |
| NO: 3 | Cadherin | KVFYSITGQGADTPPVGVFIIERETGWLKVT |
| | | EPLDRERIATYTLFSHAVSSNGNAVEDPMEI |
| | | LITVTDQNDNKPEFTQEVFKGSVMEGALPGT |
| | | SVMEVTATDADDDVNTYNAAIAYTILSQDP |
| | | ELPDKNMFTINRNTGVISVVTTGLDRESFPT |
| | | YTLVVQAADLQGEGLSTTATAVITVTDTND |
| | | NPPIFNPTTYKGQVPENEANVVITTLKVTDA |
| | | DAPNTPAWEAVYTILNDDGGQFVVTTNPVN |
| | | NDGILKTAKGLDFEAKQQYILHVAVTNVVP |
| | | FEVSLTTSTATVTVDVLDVNEAPIFVPPEKR |
| | | VEVSEDFGVGQEITSYTAQEPDTFMEQKITY |
| | | RIWRDTANWLEINPDTGAISTRAELDREDFE |
| | | HVKNSTYTALIIATDNGSPVATGTGTLLLILS |
| | | DVNDNAPIPEPRTIFFCERNPKPQVINIIDADL |
| | | PPNTSPFTAELTHGASANWTIQYNDPTQESII |
| | | LKPKMALEVGDYKINLKLMDNQNKDQVTT |
| | | LEVSVCDCEGAAGVCRKAQPVEAGLQIPAIL |
| | | GILGGILALLILILLLLLFLRRRAVVKEPLLPP |
| | | EDDTRDNVYYYDEEGGGEEDQDFDLSQLHR |
| | | GLDARPEVTRNDVAPTLMSVPRYLPRPANP |
| | | DEIGNFIDENLKAADTDPTAPPYDSLLVFDY |
| | | EGSGSEAASLSSLNSSESDKDQDYDYLNEW |
| | | GNRFKKLADMYGGGEDD |

### Antibody Binding Specificity

It is contemplated that antibodies of the present disclosure may also bind with other proteins, including, for example, recombinant proteins comprising all or a portion of KLRG1.

A KLRG1 antibody may also be a multispecific antibody, such as a bispecific or tetraspecific antibody, diabody, or similar molecule (see for instance PNAS USA 90(14), 6444-8 (1993) for a description of diabodies). Indeed, bispecific antibodies, diabodies, and the like, provided by the present invention may bind any suitable target in addition to a portion of KLRG1. For example, in one embodiment a bispecific antibody may bind KLRG1 and HER2, which would activate and redirect KLRG1+ T and NK cells to HER2+ cancer cells. In another embodiment a bispecific antibody binds KLRG1 and PD-1, which would activate T and NK cells by blocking two checkpoint receptors (KLRG1 and PD-1).

The present disclosure also provides for multispecific antibody fusion proteins which comprise an antibody moiety, for example a Fab or Fab′ fragment, with a first specificity for an antigen of interest, and further comprise at least one single domain antibody with specificity for a second antigen of interest. A multispecific antibody fusion will be capable of selectively binding to two or more antigens of interest.

In one embodiment the first and second antigen are the same antigen.

In one embodiment, an antigen of interest bound by the antibody or antigen binding fragment thereof may be a cell-associated protein, for example a cell surface protein on cells such as bacterial cells, yeast cells, T-cells, endothelial cells or tumor cells, or it may be a soluble protein. Antigens of interest may also be any medically relevant protein such as those proteins upregulated during disease or infection, for example receptors and/or their corresponding ligands. Particular examples of cell surface proteins include adhesion molecules, for example integrins such as 131 integrins e.g. VLA-4, E-selectin, P selectin or L-selectin, CD2, CD3, CD4, CD5, CD7, CD8, CD11a, CD11b, CD18, CD19, CD20, CD23, CD25, CD33, CD38, CD40, CD45, CDW52, CD69, CD134 (0X40), ICOS, BCMP7, CD137, CD27L, CDCP1, DPCR1, DPCR1, dudulin2, FLJ20584, FLJ40787, HEK2, KIAA0634, KIAA0659, KIAA1246, KIAA1455, LTBP2, LTK, MAL2, MRP2, nectin-like2, NKCCI, PTK7, RAIG1, TCAM1, SC6, BCMP101, BCMP84, BCMP11, DTD, carcinoembryonic antigen (CEA), human milk fat globulin (HMFG1 and 2), MHC Class I and MHC Class II antigens, and VEGF, and where appropriate, receptors thereof. Soluble antigens include interleukins such as IL-1, IL-2, IL-3, IL-4, IL-5, IL-6, IL-8, IL-12, IL-15, IL-16 or IL-17, viral antigens for example respiratory syncytial virus or cytomegalovirus antigens, immunoglobulins, such as IgE, interferons such as interferon a, interferon p or interferon y, tumor necrosis factor-a, tumor necrosis factor-I3, colony stimulating factors such as G-CSF or GM-CSF, and platelet derived growth factors such as PDGF-a, and PDGF-I3 and where appropriate receptors thereof. Other antigens include bacterial cell surface antigens, bacterial toxins, viruses such as influenza, EBV, HepA, B and C, bioterrorism agents, radionuclides and heavy metals, and snake and spider venoms and toxins. In a particular embodiment, the antigens of interest include IL-2, soluble IL-15 or membrane-bound IL-15Ra/IL-15 complex, and IL-12.

One skilled in the art will recognize that the antibodies of this present disclosure may be used to detect, measure, and inhibit proteins that differ somewhat from KLRG1. The antibodies can be expected to retain the specificity of binding so long as the target protein comprises a sequence which is at least about 60%, about 70%, about 80%, about 90%, about 95%, or more identical to any sequence of at least about 130, about 100, about 80, about 60, about 40, or about 20 contiguous amino acids in the sequence set forth SEQ ID NO: 1 or SEQ ID NO:2. The percent identity is determined by standard alignment algorithms such as, for example, Basic Local Alignment Tool (BLAST) described in Altshul et al. (1990) J. Mol. Biol., 215: 403-410, the algorithm of Needleman et al. (1970) J. Mol. Biol., 48: 444-453, or the algorithm of Meyers et al. (1988) Comput. Appl. Biosci., 4: 11-17.

In addition to the sequence homology analyses, epitope mapping (see, e.g., Epitope Mapping Protocols, ed. Morris, Humana Press, 1996) and secondary and tertiary structure analyses can be carried out to identify specific 3D structures assumed by the disclosed antibodies and their complexes with antigens. Such methods include, but are not limited to, X-ray crystallography (Engstom (1974) Biochem. Exp. Biol., 11:7-13) and computer modeling of virtual representations of the presently disclosed antibodies (Fletterick et al. (1986) Computer Graphics and Molecular Modeling, in Current Communications in Molecular Biology, Cold Spring Harbor Laboratory, Cold Spring Harbor, N.Y.).

### Antibody Variants

This disclosure also provides methods for obtaining an antibody specific for KLRG1. Complementarity-determining regions (CDRs) in such antibodies are not limited to the specific sequences of VH and VL identified in the Examples below, and may include variants of these sequences that retain the ability to specifically bind KLRG1. Such variants may be derived from the sequences listed in the Examples by a skilled artisan using techniques well known in the art. For example, amino acid substitutions, deletions, or additions, can be made in the framework regions (FRs) and/or in CDRs. While changes in the FRs can usually be designed to improve stability and immunogenicity of the antibody, changes in the CDRs can typically be designed to increase affinity of the antibody for its target.

Changes to FRs include, but are not limited to, humanizing a non-human derived antibody, or engineering certain framework residues that are important for antigen contact or for stabilizing the binding site, e.g., changing the class or subclass of the constant region, changing specific amino acid residues which might alter the effector function such as Fc receptor binding, e.g., as described in U.S. Pat. Nos. 5,624,821 and 5,648,260 and Lund et al. (1991) J. Immun. 147: 2657-2662 and Morgan et al. (1995) Immunology 86: 319-324, or changing the species from which the constant region is derived.

Variants of FRs also include naturally occurring immunoglobulin allotypes. Such affinity-increasing changes may be determined empirically by routine techniques that involve altering the CDR and testing the affinity antibody for its target. For example, conservative amino acid substitutions can be made within any one of the disclosed CDRs. Various alterations can be made according to the methods described, for example, in Antibody Engineering, 2^{nd} ed., Oxford University Press, ed. Borrebaeck, 1995. These include, but are not limited to, nucleotide sequences that are altered by the substitution of different codons that encode a functionally equivalent amino acid residue within the sequence, thus producing a “silent” change. For example, the nonpolar amino acids include alanine, leucine, isoleucine, valine, proline, phenylalanine, tryptophan, and methionine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Substitutes for an amino acid within the sequence may be selected from other members of the class to which the amino acid belongs. Furthermore, any native residue in the polypeptide may also be substituted with alanine (see, e.g., MacLennan et al. (1998) Acta Physiol. Scand. Suppl. 643:55-67; Sasaki et al. (1998) Adv. Biophys. 35:1-24).

The phrase “substantially as set out” means that the relevant CDR, VH, or VL domain of the disclosure will be either identical to, or have only insubstantial differences in the specified regions (e.g., a CDR) from the sequence of which is set out. Insubstantial differences include minor amino acid changes, such as substitutions of one (1) or two (2) out of any five (5) amino acids in the sequence of a specified region.

The term “KLRG1 activity” refers to one or more lymphocyte co-inhibitory activities associated with KLRG1. For example, KLRG1 activity may mean modulation of cytotoxic T and NK cell activation.

The term “modulate,” and its cognates, refer to a reduction or an increase in the activity of KLRG1 associated with activation of T cells and NK cells due to its interaction with an anti-KLRG1 antibody, wherein the reduction or increase is relative to the activity of KLRG1 in the absence of the same antibody. A reduction or an increase in activity is preferably at least about 10%, about 20%, about 30%, about 40%, about 50%, about 60%, about 70%, about 80%, about 90%, or more. When KLRG1 activity is reduced, the terms “modulatory” and “modulate” are interchangeable with the terms “inhibitory” and “inhibit.” When KLRG1 activity is increased, the terms “modulatory” and “modulate” are interchangeable with the terms “activating” and “activate.”

The term “percent (%) sequence identity” as used herein refers to the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as ClustalW, Clustal Omega, BLAST, BLAST-2, ALIGN, ALIGN-2 or Megalign (DNASTAR) software.

Antibody fragments included within the scope of the present disclosure include, for example: Fab, Fab′, F(ab′)2, and Fv fragments; domain antibodies, diabodies; vaccibodies, linear antibodies; single-chain antibody (ScFv) molecules; and multispecific (e.g., bispecific, tetraspecific) antibodies formed from antibody fragments. Such fragments can be produced by known techniques. For example, F(ab′)2 fragments can be produced by pepsin digestion of the antibody molecule, and Fab fragments can be generated by reducing the disulfide bridges of the F(ab′)2 fragments. Alternatively, Fab expression libraries can be constructed to allow rapid and easy identification of monoclonal Fab fragments with the desired specificity (see Huse et al, Science 1989 Dec. 8; 246(4935): 1275-1281).

Antibodies of the present disclosure are humanized forms of non-human (e.g., murine) antibodies and may include chimeric immunoglobulins, immunoglobulin chains or fragments thereof (such as Fv, Fab, Fab1, F(ab′)2 or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and capacity. In some instances, Fv framework residues of the human immunoglobulin can be replaced by corresponding non-human residues. Humanized antibodies may also comprise residues which are found neither in the recipient antibody, nor in the imported CDR or framework sequences. In general, the humanized antibody can comprise substantially all of at least one, and typically two, variable domains, in which all or substantially all of the CDR regions correspond to those of a non-human immunoglobulin and all or substantially all of the framework (FR) regions (i.e., the sequences between the CDR regions) are those of a human immunoglobulin consensus sequence. The humanized antibody optimally can also comprise at least a portion of an immunoglobulin constant region (Fc), typically that of a human immunoglobulin.

In certain embodiments, the VH and/or VL domains may be germlined, i.e., the framework regions (FRs) of these domains are mutated using conventional molecular biology techniques to match those produced by the germline cells. In other embodiments, the framework sequences remain diverged from the consensus germline sequences.

Methods for humanizing non-human antibodies are well known in the art. The present disclosure, and any invention(s) provided for herein, is not limited to any particular source, species of origin, or method of production.

Monoclonal antibodies used to carry out the present disclosure can be produced in a hybridoma cell line according to the technique of Kohler and Milstein, Nature 265:495 (1975). For example, a solution containing the appropriate antigen can be injected into a mouse and, after a sufficient time, the mouse sacrificed, and spleen cells obtained. The spleen cells can then be immortalized by fusing them, for example with myeloma cells or with lymphoma cells, typically in the presence of polyethylene glycol, to produce hybridoma cells. The hybridoma cells can then be grown in a suitable medium and the supernatant screened for monoclonal antibodies having the desired specificity. Monoclonal Fab fragments can be produced in E. coli by recombinant techniques known to those skilled in the art. Antibodies specific to the target polypeptide can also be obtained by phage display techniques known in the art.

Various immunoassays can be used for screening to identify antibodies having the desired specificity for the extracellular domain of KLRG1. Numerous protocols for competitive binding or immunoradiometric assays using monoclonal antibodies with established specificity are well known in the art. Such immunoassays typically involve the measurement of complex formation between an antigen and its specific antibody (e.g., antigen/antibody complex formation). A two-site, monoclonal-based immunoassay utilizing monoclonal antibodies reactive to two non- interfering epitopes on the polypeptides or peptides of this disclosure can be used as well as a competitive binding assay.

Further to the conjugates described above, anti-KLRG1 antibodies described herein can be conjugated to a solid support (e.g., beads, plates, slides, or wells formed from materials such as latex or polystyrene) in accordance with known techniques. Anti-KLRG1 antibodies described herein can likewise be conjugated to detectable groups such as radiolabels (e.g., ³⁵S, ¹²⁵I, ¹³¹I or ^{99m}Tc, which may also be attached to antibodies using conventional chemistry), enzyme labels (e.g., horseradish peroxidase, alkaline phosphatase), and fluorescence labels (e.g., fluorescein) in accordance with known techniques. Detectable labels further include chemical moieties such as biotin, which may be detected via binding to a specific cognate detectable moiety, e.g., labeled avidin. Determination of the formation of an antibody/antigen complex in the methods of this disclosure can be by detection of, for example, precipitation, agglutination, flocculation, radioactivity, color development or change, fluorescence, luminescence, etc., and such methods are well known in the art.

As described above, anti-KLRG1 antibodies described herein can be linked to another functional molecule, e.g., another peptide or protein (albumin, another antibody, etc.), toxin, radioisotope, cytotoxic or cytostatic agents. Antibodies as described herein can be conjugated to toxins. Antibody-dependent cellular phagocytosis (ADCP), antibody-dependent cellular cytotoxicity (ADCC), and complement-dependent cytotoxicity (CDC) are three well known antibody mediated mechanisms for killing target cells.

For example, the antibodies can be linked by chemical cross-linking or by recombinant methods. The antibodies may also be linked to one of a variety of nonproteinaceous polymers, e.g., polyethylene glycol, polypropylene glycol, or polyoxyalkylenes, in the manner set forth in U.S. Pat. Nos. 4,640,835, 4,496,689, 4,301,144, 4,670,417, 4,791,192, and 4,179,337. The antibodies can be chemically modified by covalent conjugation to a polymer, for example, to increase their circulating half-life. Exemplary polymers and methods to attach them are also shown in U.S. Pat. Nos. 4,766,106, 4,179,337, 4,495,285, and 4,609,546.

### PA015 Compositions

In some embodiments, an anti-KLRG1 antibody comprises a combination of any six CDRs of any one of the antibodies described herein. In some embodiments, an anti-KLRG1 antibody comprises (i) three heavy chain CDRs from Table 2 (i.e., a CDR1, a CDR2, and a CDR3), and (ii) three light chain CDRs from Table 2.

TABLE 2

| PA015 | Chothia | AbM | Kabat | Contact | IMGT |
|---|---|---|---|---|---|
| CDR- | GFSLSTFGM | GFSLSTFGMGVG | TFGMGVG | STFGMGVG | GFSLSTFGMG |
| H1 | (SEQ ID NO: 10) | (SEQ ID NO: 16) | (SEQ ID NO: 22) | (SEQ ID NO: 28) | (SEQ ID NO: 34) |
| CDR- | WWDDD | HIWWDDDKW | HIWWDDDKWYELA | WLAHIWWDDDKW | IWWDDDK |
| H2 | (SEQ ID NO: 11) | (SEQ ID NO: 17) | LKS | (SEQ ID NO: 29) | (SEQ ID NO: 35) |
| | | | (SEQ ID NO: 23) | | |
| CDR- | VIYYGSRSAYYSM | VIYYGSRSAYYSM | VIYYGSRSAYYSMD | ARVIYYGSRSAYYS | ARVIYYGSRSAYYS |
| H3 | DY | DY | Y | MD | MDY |
| | (SEQ ID NO: 12) | (SEQ ID NO: 18) | (SEQ ID NO: 24) | (SEQ ID NO: 30) | (SEQ ID NO: 36) |
| CDR- | KSSQSIVHSNAHT | KSSQSIVHSNAHT | KSSQSIVHSNAHTYL | VHSNAHTYLEWY | QSIVHSNAHTY |
| L1 | YLE | YLE | E | (SEQ ID NO: 31) | (SEQ ID NO: 37) |
| | (SEQ ID NO: 13) | (SEQ ID NO: 19) | (SEQ ID NO: 25) | | |
| CDR- | KVSNRFS | KVSNRFS | KVSNRFS | LLIYKVSNRF | KV |
| L2 | (SEQ ID NO: 14) | (SEQ ID NO: 20) | (SEQ ID NO: 26) | (SEQ ID NO: 32) | (SEQ ID NO: 38) |
| CDR- | FQGSHVPVT | FQGSHVPVT | FQGSHVPVT | FQGSHVPV | FQGSHVPVT |
| L3 | (SEQ ID NO: 15) | (SEQ ID NO: 21) | (SEQ ID NO: 27) | (SEQ ID NO: 33) | (SEQ ID NO: 39) |

There are multiple antibody variable chain numbering schemes, including those proposed by Kabat (Wu and Kabat, 1970, J Exp Med. (1970) 132:211-50.); Kabat E A, Te Wu T, Foeller C, Perry H M, Gottesman K S. Sequences of Proteins of Immunological Interest. Diane Publishing Company (1992)); Chothia (1987, Chothia C, Lesk a M. Canonical structures for the hypervariable regions of immunoglobulins. J Mol Biol. (1987) 196:901-17); Al-Lazikani et al., (1997 Standard conformations for the canonical structures of immunoglobulins. J Mol Biol. (1997) 273:927-48), Lefranc (IMGT numbering; Lefranc, 1997 Unique database numbering system for immunogenetic analysis. Immunol Today (1997) 18:509; Lefranc et al., 2005 IMGT-ONTOLOGY for immunogenetics and immunoinformatics. In Silico Biol. (2004) 4:17-29, and Honegger (Honegger and Plückthun, 2001, Yet another numbering scheme for immunoglobulin variable domains: an automatic modeling and analysis tool. J Mol Biol. (2001) 309:657-70), which makes it more complex.

Chothia and his colleagues defined CDRs based on structure, i.e. the location of the loops (Chothia and Lesk, 1987 supra; Al-Lazikani et al., 1997, supra).

Kabat and Wu originally defined the CDRs in the early 1970s by amino acid sequence variability analysis (Wu and Kabat, 1970, supra; Kabat and Wu, 1971, supra). Since then, detailed genetic analyses and three-dimensional structural analyses have more clearly defined the structural basis of the antigen combining site in terms of the HVLs/CDRs, leading to definitions of contact residues (MacCallum et al., 1996, Antibody-antigen interactions: contact analysis and binding site topography. J Mol Biol. (1996) 262:732-745; Martin and Allen, 2007, Bioinformatics tools for antibody engineering. In: Dübel S, editor. Handbook of Therapeutic Antibodies. Weinheim: Wiley-VCH Verlag GmbH (2008). p. 95-117).

The AbM definition of CDRs, which was a compromise between the Kabat and Chothia definitions, was used in the AbM modeling software (Martin and Allen, 2007, supra). Martin and his colleagues also have formed a definition of CDRs based on the actual paratope, i.e. those residues that actually contact the antigen (Martin and Allen, 2007, supra), and have developed and tested an automated application called “Abnum” for variable chain numbering and CDR determination based on a correction of the Chothia approach (Abhinandan and Martin, 2008, Analysis and prediction of VH/VL packing in antibodies. Protein Eng Des Sel. (2010) 23:689-97).

The Contact definition of CDRs is based on the observation that only 20 to 33% of the amino acids within the CDRs make direct contact with the antigen (Padlan E A. Anatomy of the antibody molecule. Mol Immunol. (1994) 31:169-217. These residues, named “Specificity Determining Residues (SDR)”, were first described by Padlan et al. (Padlan E A, Abergel C, Tipper J P. Identification of specificity-determining residues in antibodies. FASEB J OffPubl Fed Am Soc Exp Biol. (1995) 9:133-9). Their results show that these SDRs are involved in the interaction with the antigen and, in most cases, match with the most variable positions present in the CDRs. Using this SDR concept, MacCallum and co-workers suggested a new method to define the CDRs and re-named the SDRs “contact residues”. They also suggested that contact residues are more often located in the center of the paratope and, as Chothia mentioned before, non-contacting residues play a role in shaping the conformation of the CDR loops and therefore orientate the contact residues optimally for efficient and specific antigen binding.

The IMGT unique numbering for the V-REGION has allowed to redefine the limits of the Complementarity Determining Regions and Framework Regions, designated as CDR-IMGT and FR-IMGT, respectively in IMGT. The IMGT unique numbering for V-DOMAIN provides a standardized numbering for the CDR3-IMGT and FR4-IMGT of rearranged V-J-REGION and V-D-J-REGION.

A Complementarity Determining Region (CDR-IMGT) is a loop region of a V-DOMAIN (Variable (V) Domain), delimited according to the IMGT unique numbering for V domain (Lefranc M. et al. (2003) IMGT unique numbering for immunoglobulin and T cell receptor variable domains and Ig superfamily V-like domains. Dev Comp Immunol 27:55-77). There are three CDR-IMGT in a V-DOMAIN: CDR1-IMGT (loop BC), CDR2-IMGT (loop C′C″), and CDR3-IMGT (loop FG).

In a V-DOMAIN (V domain of the immunoglobulins (IG) or antibodies and T cell receptors (TR)), the amino acids of the CDR-IMGT bind to an antigen (Epitope) and confer the specificity to the IG and TR (Paratope, IMGT-ONTOLOGY, Specificity Type). The first two CDR-IMGT are part of the V-REGION (encoded by a variable (V) gene), whereas the CDR3-IMGT corresponds to the junction and results from the rearrangement between a V gene and a joining (J) gene (V-J rearrangement) or between a V gene, a diversity (D) gene, and a J gene (V-D-J rearrangement) (Immunoglobulin Synthesis).

### PA015 Sequences

One embodiment provides an antibody, preferably a monoclonal antibody, or an antigen binding fragment thereof having (i) a heavy chain variable region with at least 50%, 60%, 70%, 75%, 80%, 85%, 90%, 91%, 92%, 93%, 94%, 95%, 96%, 97%, 98%, 99%, or 100% sequence identity with amino acid sequences of SEQ ID NO: 4 and (ii) a light chain comprising a CDR-L1 (e.g., SEQ ID NO:13, SEQ ID NO:19, SEQ ID NO:25, SEQ ID NO:31, SEQ ID NO:37), CDR-L2 (e.g., SEQ ID NO:14, SEQ ID NO:20, SEQ ID NO:26, SEQ ID NO:32, SEQ ID NO:38) and CDR-L3 (e.g., SEQ ID NO:15, SEQ ID NO:21, SEQ ID NO:27, SEQ ID NO:33, SEQ ID NO:39). In a more specific embodiment, the heavy chain variable region has at least 50%, 60%, 70%, 75%, 80%, 85%, 90%, 91%, 92%, 93%, 94%, 95%, 96%, 97%, 98%, 99%, or 100% sequence identity to SEQ ID NO: 4 in regions other than the complementarity-determining region (CDR) regions such as including, but not limited to, framework (FR) regions.

One embodiment provides an antibody, preferably a monoclonal antibody, or an antigen binding fragment thereof having (i) a heavy chain variable region with at least 50%, 60%, 70%, 75%, 80%, 85%, 90%, 91%, 92%, 93%, 94%, 95%, 96%, 97%, 98%, 99%, or 100% sequence identity in regions other than the complementarity-determining region (CDR) regions such as including, but not limited to, framework (FR) regions, with amino acid sequences of SEQ ID NO: 4 and (ii) a light chain comprising a CDR-L1, a CDR-L2, and a CDR-L3, wherein the CDR-L1, CDR-L2, and CDR-L3 are SEQ ID NO:13, SEQ ID NO: 14, and SEQ ID NO: 15, respectively; SEQ ID NO: 19, SEQ ID NO:20, and SEQ ID NO:21, respectively; SEQ ID NO:25, SEQ ID NO:26, and SEQ ID NO:27, respectively; SEQ ID NO:31, SEQ ID NO:32, and SEQ ID NO:33, respectively; or SEQ ID NO:37, SEQ ID NO:38, and SEQ ID NO:39, respectively.

One embodiment provides an antibody, preferably a monoclonal antibody, or an antigen binding fragment thereof having (i) a heavy chain variable region with at least 50%, 60%, 70%, 75%, 80%, 85%, 90%, 91%, 92%, 93%, 94%, 95%, 96%, 97%, 98%, 99%, or 100% sequence identity with amino acid sequences of SEQ ID NO: 4 and (ii) a light chain comprising a CDR-L1 (ID NO:13), CDR-L2 (SEQ ID NO:14) and CDR-L3 (SEQ ID NO:15). In a more specific embodiment, the heavy chain variable region with at least 50%, 60%, 70%, 75%, 80%, 85%, 90%, 91%, 92%, 93%, 94%, 95%, 96%, 97%, 98%, 99%, or 100% sequence identity to amino acid sequences of SEQ ID NO: 4 in regions other than the complementarity-determining region (CDR) regions such as including, but not limited to, framework (FR) regions.

One embodiment provides an antibody, preferably a monoclonal antibody, or an antigen binding fragment thereof having (i) a heavy chain with at least 50%, 60%, 70%, 75%, 80%, 85%, 90%, 91%, 92%, 93%, 94%, 95%, 96%, 97%, 98%, 99%, or 100% sequence identity with amino acid sequences of SEQ ID NO: 8 and (ii) a light chain comprising a CDR-L1 (e.g., SEQ ID NO: 13, SEQ ID NO: 19, SEQ ID NO:25, SEQ ID NO:31, SEQ ID NO:37), CDR-L2 (e.g., SEQ ID NO:14, SEQ ID NO:20, SEQ ID NO:26, SEQ ID NO:32, SEQ ID NO:38) and CDR-L3 (e.g., SEQ ID NO: 15, SEQ ID NO:21, SEQ ID NO:27, SEQ ID NO:33, SEQ ID NO:39). In a more specific embodiment, the heavy chain with at least 50%, 60%, 70%, 75%, 80%, 85%, 90%, 91%, 92%, 93%, 94%, 95%, 96%, 97%, 98%, 99%, or 100% sequence identity to SEQ ID NO: 8 in regions other than the complementarity-determining region (CDR) regions such as including, but not limited to, framework (FR) regions.

One embodiment provides an antibody, preferably a monoclonal antibody, or an antigen binding fragment thereof having (i) a heavy chain with at least 50%, 60%, 70%, 75%, 80%, 85%, 90%, 91%, 92%, 93%, 94%, 95%, 96%, 97%, 98%, 99%, or 100% sequence identity in regions other than the complementarity-determining region (CDR) regions such as including, but not limited to, framework (FR) regions, with amino acid sequences of SEQ ID NO: 8 and (ii) a light chain comprising a CDR-L1, a CDR-L2, and a CDR-L3, wherein the CDR-L1, CDR-L2, and CDR-L3 are SEQ ID NO: 13, SEQ ID NO:14, and SEQ ID NO: 15, respectively; SEQ ID NO:19, SEQ ID NO:20, and SEQ ID NO:21, respectively; SEQ ID NO:25, SEQ ID NO:26, and SEQ ID NO:27, respectively; SEQ ID NO:31, SEQ ID NO:32, and SEQ ID NO:33, respectively; or SEQ ID NO:37, SEQ ID NO:38, and SEQ ID NO:39, respectively.

One embodiment provides an antibody, preferably a monoclonal antibody, or an antigen binding fragment thereof having (i) a light chain variable region with at least 50%, 60%, 70%, 75%, 80%, 85%, 90%, 91%, 92%, 93%, 94%, 95%, 96%, 97%, 98%, 99%, or 100% sequence identity with amino acid sequences of SEQ ID NO: 5 and (ii) a heavy chain comprising a CDR-H1 (e.g., SEQ ID NO:10, SEQ ID NO: 16, SEQ ID NO:22, SEQ ID NO:28, SEQ ID NO:34), CDR-H2 (e.g., SEQ ID NO:11, SEQ ID NO:17, SEQ ID NO:23, SEQ ID NO:29, SEQ ID NO:35) and CDR-H3 (e.g., SEQ ID NO:12, SEQ ID NO:18, SEQ ID NO:24, SEQ ID NO:30, SEQ ID NO:36). In a more specific embodiment, the light chain variable region has at least 50%, 60%, 70%, 75%, 80%, 85%, 90%, 91%, 92%, 93%, 94%, 95%, 96%, 97%, 98%, 99%, or 100% sequence identity to SEQ ID NO: 5 in regions other than the complementarity-determining region (CDR) regions such as including, but not limited to, framework (FR) regions.

One embodiment provides an antibody, preferably a monoclonal antibody, or an antigen binding fragment thereof having (i) a light chain variable region with at least 50%, 60%, 70%, 75%, 80%, 85%, 90%, 91%, 92%, 93%, 94%, 95%, 96%, 97%, 98%, 99%, or 100% sequence identity with amino acid sequences of SEQ ID NO: 5 and (ii) a heavy chain comprising a CDR-H1, CDR-H2, and a CDR-H3, wherein the CDR-H1, CDR-H2, CDR-H3 are SEQ ID NO:10, SEQ ID NO:11, and SEQ ID NO:12, respectively; SEQ ID NO: 16, SEQ ID NO: 17, and SEQ ID NO: 18, respectively; SEQ ID NO:22, SEQ ID NO:23, and SEQ ID NO:24, respectively; SEQ ID NO:28, SEQ ID NO:29, and SEQ ID NO:30, respectively; or SEQ ID NO:34, SEQ ID NO:35, and SEQ ID NO:36, respectively.

One embodiment provides an antibody, preferably a monoclonal antibody, or an antigen binding fragment thereof having (i) a light chain variable region with at least 50%, 60%, 70%, 75%, 80%, 85%, 90%, 91%, 92%, 93%, 94%, 95%, 96%, 97%, 98%, 99%, or 100% sequence identity with amino acid sequences of SEQ ID NO: 5 and (ii) a heavy chain comprising CDR-H1 (SEQ ID NO:10), CDR-H2 (SEQ ID NO:11) and CDR-H3 (SEQ ID NO:12). In a more specific embodiment, the light chain variable region has at least 50%, 60%, 70%, 75%, 80%, 85%, 90%, 91%, 92%, 93%, 94%, 95%, 96%, 97%, 98%, 99%, or 100% sequence identity to SEQ ID NO:5 in regions other than the complementarity-determining region (CDR) regions such as including, but not limited to, framework (FR) regions.

One embodiment provides an antibody, preferably a monoclonal antibody, or an antigen binding fragment thereof having (i) a light chain with at least 50%, 60%, 70%, 75%, 80%, 85%, 90%, 91%, 92%, 93%, 94%, 95%, 96%, 97%, 98%, 99%, or 100% sequence identity with amino acid sequences of SEQ ID NO: 9 and (ii) a heavy chain comprising a CDR-H1 (e.g., SEQ ID NO:10, SEQ ID NO:16, SEQ ID NO:22, SEQ ID NO:28, SEQ ID NO:34), CDR-H2 (e.g., SEQ ID NO:11, SEQ ID NO: 17, SEQ ID NO:23, SEQ ID NO:29, SEQ ID NO:35) and CDR-H3 (e.g., SEQ ID NO: 12, SEQ ID NO: 18, SEQ ID NO:24, SEQ ID NO:30, SEQ ID NO:36). In a more specific embodiment the light chain has at least 50%, 60%, 70%, 75%, 80%, 85%, 90%, 91%, 92%, 93%, 94%, 95%, 96%, 97%, 98%, 99%, or 100% sequence identity to SEQ ID NO: 9 in regions other than the complementarity-determining region (CDR) regions such as including, but not limited to, framework (FR) regions.

A particular embodiment provides an antibody, preferably a monoclonal antibody, or an antigen binding fragment thereof having (i) a light chain with at least 50%, 60%, 70%, 75%, 80%, 85%, 90%, 91%, 92%, 93%, 94%, 95%, 96%, 97%, 98%, 99%, or 100% sequence identity in regions other than the complementarity-determining region (CDR) regions such as including, but not limited to, framework (FR) regions, with amino acid sequences of SEQ ID NO: 9 and (ii) a heavy chain comprising a CDR-H1, CDR-H2, and a CDR-H3, wherein the CDR-H1, CDR-H2, CDR-H3 are SEQ ID NO: 10, SEQ ID NO: 11, and SEQ ID NO:12, respectively; SEQ ID NO:16, SEQ ID NO: 17, and SEQ ID NO: 18, respectively; SEQ ID NO:22, SEQ ID NO:23, and SEQ ID NO:24, respectively; SEQ ID NO:28, SEQ ID NO:29, and SEQ ID NO:30, respectively; or SEQ ID NO:34, SEQ ID NO:35, and SEQ ID NO:36, respectively.

One embodiment provides an antibody, preferably a monoclonal antibody, or an antigen binding fragment thereof having (i) a light chain with at least 50%, 60%, 70%, 75%, 80%, 85%, 90%, 91%, 92%, 93%, 94%, 95%, 96%, 97%, 98%, 99%, or 100% sequence identity with amino acid sequences of SEQ ID NO: 9 and (ii) a heavy chain comprising CDR-H1 (SEQ ID NO:10), CDR-H2 (SEQ ID NO:11) and CDR-H3 (SEQ ID NO:12). In a more specific embodiment, the light chain has at least 50%, 60%, 70%, 75%, 80%, 85%, 90%, 91%, 92%, 93%, 94%, 95%, 96%, 97%, 98%, 99%, or 100% sequence identity with amino acid sequences of SEQ ID NO: 9 in regions other than the complementarity-determining region (CDR) regions such as including, but not limited to, framework (FR) regions.

One embodiment provides an antibody, preferably a monoclonal antibody, or an antigen binding fragment thereof having (i) a light chain with at least 50%, 60%, 70%, 75%, 80%, 85%, 90%, 91%, 92%, 93%, 94%, 95%, 96%, 97%, 98%, 99%, or 100% sequence identity with amino acid sequences of SEQ ID NO: 9 and (ii) a heavy chain with at least 50%, 60%, 70%, 75%, 80%, 85%, 90%, 91%, 92%, 93%, 94%, 95%, 96%, 97%, 98%, 99%, or 100% sequence identity with amino acid sequences of SEQ ID NO: 8. In a more specific embodiment, the light chain has at least 50%, 60%, 70%, 75%, 80%, 85%, 90%, 91%, 92%, 93%, 94%, 95%, 96%, 97%, 98%, 99%, or 100% sequence identity with amino acid sequences of SEQ ID NO: 9 in regions other than the complementarity-determining region (CDR) regions such as including, but not limited to, framework (FR) regions; and heavy chain with at least 50%, 60%, 70%, 75%, 80%, 85%, 90%, 91%, 92%, 93%, 94%, 95%, 96%, 97%, 98%, 99%, or 100% sequence identity with amino acid sequences of SEQ ID NO: 8 in regions other than the complementarity-determining region (CDR) regions such as including, but not limited to, framework (FR) regions.

One embodiment provides an antibody, preferably a monoclonal antibody, or an antigen binding fragment thereof having (i) a light chain variable region with at least 50%, 60%, 70%, 75%, 80%, 85%, 90%, 91%, 92%, 93%, 94%, 95%, 96%, 97%, 98%, 99%, or 100% sequence identity with amino acid sequences of SEQ ID NO: 9 in regions other than the complementarity-determining region (CDR) regions such as including, but not limited to, framework (FR) regions and (ii) a heavy chain with at least 50%, 60%, 70%, 75%, 80%, 85%, 90%, 91%, 92%, 93%, 94%, 95%, 96%, 97%, 98%, 99%, or 100% sequence identity in regions other than the complementarity-determining region (CDR) regions such as including, but not limited to, framework (FR) regions with amino acid sequences of SEQ ID NO: 8.

One embodiment provides an antibody, preferably a monoclonal antibody, or an antigen binding fragment thereof having (i) a light chain variable region containing CDRs (CDR-L1, CDR-L2, CDR-L3) having amino acid sequences SEQ ID Nos: 13, 14, and 15, respectively; (ii) a heavy chain variable region containing CDRs (CDR-H1, CDR-H2, CDR-H3) having amino acid sequences SEQ ID Nos: 10, 11, and 12, respectively; (iii) a heavy chain constant region with at least 50%, 60%, 70%, 75%, 80%, 85%, 90%, 91%, 92%, 93%, 94%, 95%, 96%, 97%, 98%, 99%, or 100% sequence identity with the amino acid sequence of SEQ ID NO:6 and (iv) a light chain constant region with at least 50%, 60%, 70%, 75%, 80%, 85%, 90%, 91%, 92%, 93%, 94%, 95%, 96%, 97%, 98%, 99%, or 100% sequence identity with the amino acid sequence of SEQ ID NO:7.

One embodiment provides an antibody, preferably a monoclonal antibody, or an antigen binding fragment thereof having (i) a light chain variable region containing CDRs (CDR-L1, CDR-L2, CDR-L3) having amino acid sequences SEQ ID Nos: 13, 14, and 15, respectively; (ii) a heavy chain variable region containing CDRs (CDR-H1, CDR-H2, CDR-H3) having amino acid sequences SEQ ID Nos: 10, 11, and 12, respectively; (iii) a heavy chain with at least 50%, 60%, 70%, 75%, 80%, 85%, 90%, 91%, 92%, 93%, 94%, 95%, 96%, 97%, 98%, 99%, or 100% sequence identity with the amino acid sequence of SEQ ID NO:8 and (iv) a light chain with at least 50%, 60%, 70%, 75%, 80%, 85%, 90%, 91%, 92%, 93%, 94%, 95%, 96%, 97%, 98%, 99%, or 100% sequence identity with the amino acid sequence of SEQ ID NO:9.

In some embodiments, the antibody, or an antigen-binding fragment thereof, comprises (i) a heavy chain variable region that includes at least one, two or three CDR (e.g., CDR-H1, CDR-H2, or CDR-H3), or a portion thereof, having the amino acid sequence of SEQ ID Nos: 10, 11, or 12, respectively, and (ii) a light chain variable region that includes at least one, two or three CDR (e.g., CDR-L1, CDR-L2, or CDR-L3), or a portion thereof, having the amino acid sequence of SEQ ID Nos: 13, 14, or 15, respectively.

One embodiment provides an antibody, preferably a monoclonal antibody, or an antigen binding fragment thereof having a light chain variable region with at least 50%, 60%, 70%, 75%, 80%, 85%, 90%, 91%, 92%, 93%, 94%, 95%, 96%, 97%, 98%, 99%, or 100% sequence identity in regions other than the complementarity-determining region (CDR) regions such as including, but not limited to, framework (FR) regions, with the amino acid sequence of SEQ ID NO: 5

One embodiment provides an antibody, preferably a monoclonal antibody, or an antigen binding fragment thereof having a heavy chain variable region with at least 50%, 60%, 70%, 75%, 80%, 85%, 90%, 91%, 92%, 93%, 94%, 95%, 96%, 97%, 98%, 99%, or 100% sequence identity in regions other than the CDR regions such as including but not limited to FR regions, with the amino acid sequence of SEQ ID NO: 4.

One embodiment provides an antibody, preferably a monoclonal antibody, or an antigen binding fragment thereof, having (i) a light chain variable region with at least 50%, 60%, 70%, 75%, 80%, 85%, 90%, 91%, 92%, 93%, 94%, 95%, 96%, 97%, 98%, 99%, or 100% sequence identity in regions other than the CDR regions such as including but not limited to framework (FR) regions, with the amino acid sequence of SEQ ID NO: 5 and (ii) a heavy chain variable region with at least 50%, 60%, 70%, 75%, 80%, 85%, 90%, 91%, 92%, 93%, 94%, 95%, 96%, 97%, 98%, 99%, or 100% sequence identity in regions other than the CDR regions such as including, but not limited to, FR regions, with the amino acid sequence of SEQ ID NO: 4.

One embodiment provides an antibody, preferably a monoclonal antibody, or antigen binding fragment thereof that has (i) a light chain variable region containing CDRs (CDR1, CDR2, CDR3) having amino acid sequences SEQ ID NOs: 13, 14 and 15, respectively; (ii) a heavy chain variable region containing CDRs (CDR1, CDR2, CDR3) having amino acid sequences SEQ ID NOs:10, 11 and 12, respectively; (iii) a heavy chain constant region with at least 50%, 60%, 70%, 75%, 80%, 85%, 90%, 91%, 92%, 93%, 94%, 95%, 96%, 97%, 98%, 99%, or 100% sequence identity with the amino acid sequence of SEQ ID NO: 6 and (iv) a light chain constant region with at least 50%, 60%, 70%, 75%, 80%, 85%, 90%, 91%, 92%, 93%, 94%, 95%, 96%, 97%, 98%, 99%, or 100% sequence identity with the amino acid sequence of SEQ ID NO: 7.

In certain aspects of all of the aforementioned embodiments, when a light or heavy chain, variable region, or constant region is stated to have certain percentages of sequence identity with a reference SEQ ID NO, such identity is at least 90%, 91%, 92%, 93%, 94%, 95%, 96%, 97%, 98%, 99%, or 100%.

### Immunotoxins

In some aspects, the antibodies and/or antigen binding fragments thereof provided for by the instant disclosure are conjugated to a toxic agent. Immunoconjugates which include one or more cytotoxins are referred to as “immunotoxins.” Antibodies conjugated to a cytotoxic agent, drug, or the like are also known as antibody-drug conjugates (ADC). An immunoconjugate may have a half-life of sufficient periods of time for the antibody-drug conjugate to be internalized, degraded, and induce cell killing by the released toxin. A cytotoxin or cytotoxic agent can include any agent that is detrimental to (e.g., kills) cells. Suitable cytotoxic agents for forming immunoconjugates of the present disclosure include taxol, tubulysins, duostatins, cytochalasin B, gramicidin D, ethidium bromide, emetine, mitomycin, etoposide, tenoposide, vincristine, vinblastine, colchicin, doxorubicin, daunorubicin, dihydroxy anthracin dione, maytansine or an analog or derivative thereof, mitoxantrone, mithramycin, actinomycin D, 1-dehydrotestosterone, glucocorticoids, procaine, tetracaine, lidocaine, propranolol, and puromycin; calicheamicin or analogs or derivatives thereof, antimetabolites (such as methotrexate, 6-mercaptopurine, 6-thioguanine, cytarabine, fludarabin, 5-fluorouracil, decarbazine, hydroxyurea, asparaginase, gemcitabine, cladribine), alkylating agents (such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin; as well as duocarmycin A, duocarmycin SA, CC-1065 (a.k.a. rachelmycin), or analogs or derivatives of CC-1065), dolastatin, auristatin, pyrrolo[2,1-c][1,4]benzodiazepins (PDBs), indolinobenzodiazepine (IGNs) or analogues thereof, antibiotics (such as dactinomycin (formerly actinomycin), bleomycin, daunorubicin (formerly daunomycin), doxorubicin, idarubicin, mithramycin, mitomycin, mitoxantrone, plicamycin, anthramycin (AMC)), anti-mitotic agents (e.g., tubulin-targeting agents), such as diphtheria toxin and related molecules (such as diphtheria A chain and active fragments thereof and hybrid molecules); ricin toxin (such as ricin A or a deglycosylated ricin A chain toxin), cholera toxin, a Shiga-like toxin (SLT-I, SLT-II, SLT-IIV), LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins (PAPI, PAPII, and PAP-S), Momordica charantia inhibitor, curcin, crotin, Sapaonaria officinalis inhibitor, gelonin, mitogellin, restrictocin, phenomycin, and enomycin toxins. Other suitable conjugated molecules include antimicrobial/lytic peptides such as CLIP, Magainin 2, mellitin, Cecropin, and P18; ribonuclease (RNase), DNase I, Staphylococcal enterotoxin-A, pokeweed antiviral protein, diphtheria toxin, and Pseudomonas endotoxin.

### Supplemental Therapeutic Agents

The antibodies of the present disclosure, including fragments thereof and conjugates thereof, can optionally be delivered to a patient in conjunction with other therapeutic agents. The additional therapeutic agents can be delivered before, concurrently with, or after the antibodies of the present disclosure. As used herein, the word “concurrently” means sufficiently close in time to produce a combined effect (that is, concurrently can be simultaneously, or it can be two or more events occurring within a short time period before or after each other).

In some embodiments the antibodies of the present disclosure can be administered in conjunction with anti-cancer agents, such as: 1) vinca alkaloids (e.g., vinblastine, vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide). In some embodiments the antibodies of the present disclosure can be administered in conjunction with anti-angiogenesis agents, such as antibodies to VEGF (e.g., bevacizumab (AVASTIN), ranibizumab (LUCENTIS)) and other promoters of angiogenesis (e.g., bFGF, angiopoietin-1), antibodies to alpha-v/beta-3 vascular integrin (e.g., VITAXIN), angiostatin, endostatin, dalteparin, ABT-510, CNGRC peptide (SEQ ID NO: 43) TNF alpha conjugate, cyclophosphamide, combretastatin A4 phosphate, dimethylxanthenone acetic acid, docetaxel, lenalidomide, enzastaurin, paclitaxel, paclitaxel albumin-stabilized nanoparticle formulation (Abraxane), soy isoflavone (Genistein), tamoxifen citrate, thalidomide, ADH-1 (EXHERIN), AG-013736, AMG-706, AZD2171, sorafenib tosylate, BMS-582664, CHIR-265, pazopanib, PI-88, vatalanib, everolimus, suramin, sunitinib malate, XL184, ZD6474, ATN-161, cilengitide, and celecoxib.

In some embodiments, the antibodies of the present disclosure can be administered in conjunction with immunosuppressive agents including, for example, cyclosporine A, rapamycin, glucocorticoids, azathioprine, mizoribine, aspirin derivatives, hydroxychloroquine, methotrexate, cyclophosphamide and FK506 (tacrolimus).

### Nucleic Acids, Cloning and Expression Systems

The present disclosure further provides isolated nucleic acids encoding the disclosed antibodies. The nucleic acids may comprise DNA or RNA and may be wholly or partially synthetic or recombinant. Reference to a nucleotide sequence as set out herein encompasses a DNA molecule with the specified sequence, and encompasses a RNA molecule with the specified sequence in which U is substituted for T, unless context requires otherwise.

The nucleic acids provided herein comprise a coding sequence for a CDR, a VH domain, and/or a VL domain disclosed herein. The present disclosure also provides constructs in the form of plasmids, vectors, phagemids, transcription or expression cassettes which comprise at least one nucleic acid encoding a CDR, a VH domain, and/or a VL domain disclosed here. The disclosure further provides a host cell which comprises one or more constructs as above.

In some embodiments, the coding sequence for the heavy chain is the nucleotide sequence of SEQ ID NO:40.

In some embodiments, the coding sequence for the light chain is the nucleotide sequence of SEQ ID NO:41.

Systems for cloning and expression of a polypeptide in a variety of different host cells are well known in the art. For cells suitable for producing antibodies, see Gene Expression Systems, Academic Press, eds. Fernandez et al., 1999. Briefly, suitable host cells include bacteria, plant cells, mammalian cells, and yeast and baculovirus systems. Mammalian cell lines available in the art for expression of a heterologous polypeptide include Chinese hamster ovary cells, HeLa cells, baby hamster kidney cells, NSO mouse myeloma cells, and many others. A common bacterial host is E. coli. Any protein expression system compatible with the invention may be used to produce the disclosed antibodies. Suitable expression systems include transgenic animals described in Gene Expression Systems, Academic Press, eds. Fernandez et al., 1999.

Suitable vectors can be chosen or constructed, so that they contain appropriate regulatory sequences, including promoter sequences, terminator sequences, polyadenylation sequences, enhancer sequences, marker genes and other sequences as appropriate. Vectors may be plasmids or viral, e.g., phage, or phagemid, as appropriate. For further details see, for example, Sambrook et al., Molecular Cloning: A Laboratory Manual, 2nd ed., Cold Spring Harbor Laboratory Press, 1989. Many known techniques and protocols for manipulation of nucleic acid, for example, in preparation of nucleic acid constructs, mutagenesis, sequencing, introduction of DNA into cells and gene expression, and analysis of proteins, are described in detail in Current Protocols in Molecular Biology, 2nd Edition, eds. Ausubel et al., John Wiley & Sons, 1992.

A further aspect of the disclosure provides a host cell comprising a nucleic acid as disclosed here. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage. The introduction of the nucleic acid into the cells may be followed by causing or allowing expression from the nucleic acid, e.g., by culturing host cells under conditions for expression of the gene.

### Methods of Treatment

The disclosed anti-KLRG1 antibodies are capable of modulating the KLRG1-associated immune responses. In particular embodiments, the activation of cytotoxic T and NK cells is mediated by modulation of KLRG1 signaling. The disclosed antibodies can act as either agonists or antagonists of KLRG1, depending on the method of their use. The antibodies can be used to prevent, diagnose, or treat medical disorders in mammals, especially, in humans. Antibodies of the disclosure can also be used for isolating KLRG1 or KLRG1-expressing cells. Furthermore, the antibodies can be used to treat a subject at risk of, or susceptible to, a disorder, or having a disorder associated with aberrant KLRG1 expression or function.

Antibodies of the disclosure can be used in circumstances where modulation of cytotoxic T and NK cell activation may be desirable, for example, in certain types of cancers and infectious diseases.

Under certain circumstances, it may be desirable to elicit or enhance a patient's immune response in order to treat cancer or an infectious disease. The disorders being treated or prevented by the disclosed methods include, but are not limited to, infections with microbes (e.g. bacteria), viruses (e.g., systemic viral infections such as influenza, viral skin diseases such as herpes or shingles), or parasites; and cancer (e.g., melanoma and prostate cancers). Cytotoxic T and NK cell activation with the anti-KLRG1 antibodies disclosed herein enhance T and NK cell responses. In such cases, antibodies act as antagonists of KLRG1. Thus, in some embodiments, the antibodies can be used to inhibit or reduce the downregulatory activity associated with KLRG1, i.e., the activity associated with downregulation of cytotoxic T and NK cell activation.

As demonstrated in the Examples, a blockade of KLRG1/E-cadherin interaction with antagonizing anti-KLRG1 antibodies leads to enhanced T cell proliferative responses and IFNy secretion by these cells, consistent with a downregulatory role for the KLRG1 pathway in cytotoxic T and NK cell activation. In various embodiments, the antibodies inhibit binding of E-cadherin to KLRG1 with an IC50 of less than about 50 nM, and more preferably less than about 40, 30, 20, 10, or 5 nM. Inhibition of E-cadherin binding can be measured using techniques known in the art.

The antibodies or antibody compositions of the present disclosure are administered in therapeutically effective amounts. Generally, a therapeutically effective amount may vary with the subject's age, condition, and sex, as well as the severity of the medical condition of the subject. A therapeutically effective amount of antibody ranges from about 0.001 to about 30 mg/kg body weight, preferably from about 0.01 to about 25 mg/kg body weight, from about 0.1 to about 20 mg/kg body weight, or from about 1 to about 10 mg/kg body weight. The dosage may be adjusted, as necessary, to suit observed effects of the treatment.

The appropriate dose is chosen based on clinical indications by a treating physician. The antibodies may be given as a bolus dose, to maximize the circulating levels of antibodies for the greatest length of time after the dose. Continuous infusion may also be used after the bolus dose.

Immune cells (e.g., T cells or NK cells) can also be isolated from a patient and incubated ex vivo with antibodies of the disclosure. In some embodiments, T cell and NK cell activation can be modulated by removing immune cells from a subject, contacting the immune cells in vitro with an anti-KLRG1 antibody of the disclosure. In such embodiments, the anti-KLRG1 antibody may be used in a multivalent form such that KLRG1 molecules on the surface of an immune cell become “crosslinked” upon binding to such antibodies. For example, the anti-KLRG1 antibodies can be bound to solid support, such as beads, or crosslinked via a secondary antibody. The immune cells may be then isolated using methods known in the art and reimplanted into the patient.

The antibodies of the disclosure may also be used to detect the presence of KLRG1 in biological samples. The amount of KLRG1 detected may be correlated with the expression level of KLRG1, which, in turn, is correlated with the activation status of immune cells (e.g., activated T cells or NK cells) in the subject.

Detection methods that employ antibodies are well known in the art and include, for example, ELISA, radioimmunoassay, immunoblot, Western blot, immunofluorescence, immunoprecipitation. The antibodies may be provided in a diagnostic kit that incorporates one or more of these techniques to detect KLRG1. Such a kit may contain other components, packaging, instructions, or other material to aid in the detection of the protein.

Where the antibodies are intended for diagnostic purposes, it may be desirable to modify them, for example, with a ligand group (such as biotin) or a detectable marker group (such as a fluorescent group, a radioisotope or an enzyme). If desired, the antibodies of the disclosure may be labeled using conventional techniques. Suitable detectable labels include, for example, fluorophores, chromophores, radioactive atoms, electron-dense reagents, enzymes, and ligands having specific binding partners. Enzymes are typically detected by their activity. For example, horseradish peroxidase can be detected by its ability to convert tetramethylbenzidine (TMB) to a blue pigment, quantifiable with a spectrophotometer. For detection, suitable binding partners include, but are not limited to, biotin and avidin or streptavidin, IgG and protein A, and the numerous receptor-ligand couples known in the art. Other permutations and possibilities will be readily apparent to those of ordinary skill in the art, and are considered as equivalents within the scope of the instant disclosure.

Antibodies of the disclosure can be used in screening methods to identify inhibitors of the KLRG1 pathway effective as therapeutics. In such a screening assay, a first binding mixture is formed by combining KLRG1 and an antibody of the disclosure; and the amount of binding in the first binding mixture (MO) is measured. A second binding mixture is also formed by combining KLRG1, the antibody, and the compound or agent to be screened, and the amount of binding in the second binding mixture (M1) is measured. A compound to be tested may be another anti-KLRG1 antibody, as illustrated in the Examples. The amounts of binding in the first and second binding mixtures are then compared, for example, by calculating the M1/M0 ratio. The compound or agent is considered to be capable of modulating a KLRG1-associated downregulation of immune responses if a decrease in binding in the second binding mixture as compared to the first binding mixture is observed.

The formulation and optimization of binding mixtures is within the level of skill in the art, such binding mixtures may also contain buffers and salts necessary to enhance, or to optimize, binding, and additional control assays may be included in the screening assay of the disclosure. Compounds found to reduce the KLRG1-antibody binding by at least about 10% (i.e., M1/MO<0.9), preferably greater than about 30% may thus be identified and then, if desired, secondarily screened for the ability to ameliorate a disorder in other assays or animal models as described below. The strength of the binding between KLRG1 and an antibody can be measured using, for example, an enzyme-linked immunoadsorption assay (ELISA), radio-immunoassay (RIA), surface plasmon resonance-based technology (e.g., Biacore), all of which are techniques well known in the art.

The compound may then be tested in vitro as described in the Examples or in an animal model. Preliminary doses as, for example, determined according to animal tests, and the scaling of dosages for human administration is performed according to art-accepted practices. Toxicity and therapeutic efficacy can be determined by standard pharmaceutical procedures in cell cultures or experimental animals. The data obtained from the cell culture assays or animal studies can be used in formulating a range of dosages for use in humans.

Therapeutically effective dosages achieved in one animal model can be converted for use in another animal, including humans, using conversion factors known in the art (see, e.g., Freireich et al. (1966) Cancer Chemother. Reports, 50(4): 219-244).

### Pharmaceutical Compositions

The disclosure provides pharmaceutical compositions comprising anti-KLRG1 antibodies. Such compositions may be suitable for pharmaceutical use and administration to patients. The compositions typically comprise one or more antibodies of the present disclosure and a pharmaceutically acceptable excipient. The phrase “pharmaceutically acceptable excipient” includes any and all solvents, dispersion media, coatings, antibacterial agents and antifungal agents, isotonic agents, and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

A pharmaceutical composition of the disclosure is formulated to be compatible with its intended route of administration. Methods to accomplish the administration are known to those of ordinary skill in the art. The administration may, for example, be intravenous, intraperitoneal, intramuscular, intracavity, subcutaneous or transdermal. It may also be possible to obtain compositions which may be topically or orally administered, or which may be capable of transmission across mucous membranes.

Solutions or suspensions used for intradermal or subcutaneous application typically include one or more of the following components: a sterile diluent such as water for injection, saline solution, fixed oils, polyethylene glycols, glycerin, propylene glycol, or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose. The pH can be adjusted with acids or bases, such as hydrochloric acid or sodium hydroxide. Such preparations may be enclosed in ampoules, disposable syringes or multiple dose vials made of glass or plastic.

Pharmaceutical compositions suitable for injection include sterile aqueous solutions or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringeability exists. It should be stable under the conditions of manufacture and storage and must be preserved against the contaminating action of microorganisms such as bacteria and fungi. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars; polyalcohols, such as mannitol, sorbitol, and sodium chloride in the composition. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and/or by the use of surfactants. Prolonged absorption of the injectable compositions can be brought about by including in the composition an agent which delays absorption, for example, aluminum monostearate, and gelatin.

### Administration and Dosage

Methods to accomplish administration are known to those of ordinary skill in the art. The administration may, for example, be parental administration (e.g., intravenous, intraperitoneal, intramuscular, subcutaneous).

Systemic administration can also be by transmucosal or transdermal means. For transmucosal or transdermal administration, penetrants appropriate to the barrier to be permeated are used in the formulation. Such penetrants are generally known in the art, and include, for example, detergents, bile salts, and fusidic acid derivatives. Transmucosal administration may be accomplished, for example, through the use of lozenges, nasal sprays, inhalers, or suppositories; For example, in case of antibodies that comprise the Fc portion, compositions may be capable of transmission across mucous membranes in intestine, mouth, or lungs (e.g., via the FcRn receptor-mediated pathway as described in U.S. Pat. No. 6,030,613). For transdermal administration, the active compounds may be formulated into ointments, salves, gels, or creams as generally known in the art. For administration by inhalation, the antibodies may be delivered in the form of an aerosol spray from pressured container or dispenser, which contains a suitable propellant, e.g., a gas such as carbon dioxide, or a nebulizer.

In certain embodiments, the presently disclosed antibodies are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions containing the presently disclosed antibodies can also be used as pharmaceutically acceptable carriers. These can be prepared according to methods known to those skilled in the art, for example, as described in U.S. Pat. No. 4,522,811.

It may be advantageous to formulate parenteral compositions in a dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of the composition of the disclosure can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50.

Compositions that exhibit large therapeutic indices are preferred. For any composition used in the present disclosure, the therapeutically effective dose can be estimated initially from cell culture assays. Examples of suitable bioassays include DNA replication assays, cytokine release assays, transcription-based assays, KLRG1/cadherin binding assays, immunological assays other assays as, for example, described in the Examples. The data obtained from the cell culture assays and animal studies can be used in formulating a range of dosage for use in humans. A dose may be formulated in animal models to achieve a circulating plasma concentration range that includes the IC50 (i.e., the concentration of the antibody which achieves a half-maximal inhibition of symptoms). Circulating levels in plasma may be measured, for example, by high performance liquid chromatography. The effects of any particular dosage can be monitored by a suitable bioassay. The dosage lies preferably within a range of circulating concentrations with little or no toxicity. The dosage may vary depending upon the dosage form employed and the route of administration utilized.

The following Examples do not in any way limit the scope of the disclosure. One of ordinary skill in the art will recognize the numerous modifications and variations that may be performed without altering the spirit or scope of the present disclosure. Such modifications and variations are encompassed within the scope of the disclosure. The entire contents of all references, patents, and published patent applications cited throughout this application are herein incorporated by reference.

Recombinant proteins were produced by standard molecular cloning and expression protocols include human KLRG1 ECD (SEQ ID NO:1), cynomolgus KLRG1 ECD (SEQ ID NO:2), and human E-cadherin (SEQ ID NO:3), whose amino acid sequences are known in art and are disclosed in the PCT publication, WO2020060781, which is hereby by incorporated by reference in its entirety. Recombinant proteins were produced as FC fusion or as HIS tagged versions by cloning the respective cDNA into pCDNA4 vector (Invitrogen) and transient transfection in mammalian HEK293. Purification of the expressed proteins took place by chromatography using Protein A affinity resin for the FC fusion versions and Nickel-NTA resin for HIS tagged proteins. All purified proteins were characterized by SDS-PAGE electrophoresis to verify purity and molecular weight.

EXEMPLARY SEQUENCES

PA015 HEAVY CHAIN VARIABLE REGION SEQUENCE
>PA015_VH (SEQ ID NO: 4)
QVTLRESGPALVKPTQTLTLTCTVSGFSLSTFGMGVGWIRQPPGKALEWLAHIW
WDDDKWYELALKSRLTISKDTSKNQVVLTITNMDPVDTATYYCARVIYYGSRSA
YYSMDYWGQGTTVTVSS
PA015 LIGHT CHAIN VARIABLE REGION SEQUENCE
>PA015_VL (SEQ ID NO: 5)
DVVMTQTPLSLSVTPGQPASISCKSSQSIVHSNAHTYLEWYLQKPGQSPQLLIYK
VSNRFSGVPDRFSGSGSGTDFTLKISRVEAEDVGVYYCFQGSHVPVTFGQGTKLE
IKRTV
PA015 HEAVY CHAIN CONSTANT REGION SEQUENCE
>PA015_Heavy chain constant region (SEQ ID NO: 6)
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCP
PCPAPEAAGAPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSL
SPGK
PA015 LIGHT CHAIN CONSTANT REGION SEQUENCE
>PA015_Light chain constant region (SEQ ID NO: 7)
AAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
PA015 HEAVY CHAIN SEQUENCE
>PA015_HC (SEQ ID NO: 8)
QVTLRESGPALVKPTQTLTLTCTVSGFSLSTFGMGVGWIRQPPGKALEWLAHIW
WDDDKWYELALKSRLTISKDTSKNQVVLTITNMDPVDTATYYCARVIYYGSRSA
YYSMDYWGQGTTVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNT
KVDKKVEPKSCDKTHTCPPCPAPEAAGAPSVFLFPPKPKDTLMISRTPEVTCVVV
DVSHEDPEVKFNWYVDGVEVHNAKTKPREEQYNSTYRVVSVLTVLHQDWLNG
KEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSC
SVMHEALHNHYTQKSLSLSPGK
PA015 LIGHT CHAIN SEQUENCE
>PA015_LC (SEQ ID NO: 9)
DVVMTQTPLSLSVTPGQPASISCKSSQSIVHSNAHTYLEWYLQKPGQSPQLLIYK
VSNRFSGVPDRFSGSGSGTDFTLKISRVEAEDVGVYYCFQGSHVPVTFGQGTKLE
IKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
>PA015_VH_CDR1 (SEQ ID NO: 10)-Chothia Format
GFSLSTFGM
>PA015_VH_CDR2 (SEQ ID NO: 11)-Chothia Format
WWDDD
>PA015_VH_CDR3 (SEQ ID NO: 12)-Chothia Format
VIYYGSRSAYYSMDY
>PA015_VL_CDR1 (SEQ ID NO: 13)-Chothia Format
KSSQSIVHSNAHTYLE
>PA015_VL_CDR2 (SEQ ID NO: 14)-Chothia Format
KVSNRFS
>PA015_VL_CDR3 (SEQ ID NO: 15)-Chothia Format
FQGSHVPVT
>PA015_HC (SEQ ID NO: 40)
ATGGGCTGGTCCTGCATCATTCTGTTTCTGGTGGCTACCGCCACCGGCGCTCATTCTCAAGTGACACTGAGAGAGTC
TGGCCCCGCTCTGGTCAAGCCTACACAGACCCTGACACTGACCTGCACCGTGTCTGGCTTCTCCCTGTCTACCTTTG
GCATGGGCGTCGGCTGGATTAGACAGCCTCCTGGAAAAGCCCTGGAATGGCTGGCCCACATTTGGTGGGACGACGAC
AAGTGGTACGAGCTGGCTCTGAAGTCCCGGCTGACCATCTCCAAGGACACCTCCAAGAATCAGGTGGTGCTGACAAT
CACCAACATGGACCCTGTGGACACCGCCACCTACTACTGCGCCAGAGTGATCTACTACGGCTCCAGATCCGCCTACT
ACTCCATGGATTATTGGGGCCAGGGCACCACCGTGACCGTGTCCTCTGCTTCTACAAAGGGCCCCTCTGTGTTCCCT
CTGGCTCCTTCCTCTAAATCCACCTCTGGCGGAACCGCTGCTCTGGGCTGTCTCGTGAAGGATTACTTCCCTGAGCC
TGTGACAGTGTCCTGGAATAGCGGTGCTCTGACATCCGGCGTGCACACCTTTCCAGCTGTGCTGCAGTCCTCTGGCC
TGTACTCTCTGTCCTCTGTCGTGACAGTGCCTTCCAGCTCTCTGGGCACCCAGACCTACATCTGCAACGTGAACCAC
AAGCCTTCCAACACCAAGGTGGACAAGAAGGTGGAACCCAAGTCCTGCGACAAGACCCACACCTGTCCTCCATGTCC
TGCTCCAGAAGCTGCTGGCGCTCCCTCCGTGTTTCTGTTCCCTCCAAAGCCTAAGGACACCCTGATGATCTCTCGGA
CCCCTGAAGTGACCTGCGTGGTGGTGGATGTGTCTCACGAGGATCCCGAAGTGAAGTTCAATTGGTACGTGGACGGC
GTGGAAGTGCACAACGCCAAGACCAAGCCTAGAGAGGAACAGTACAACTCCACCTACAGAGTGGTGTCCGTGCTGAC
CGTGCTGCACCAGGATTGGCTGAACGGCAAAGAGTACAAGTGCAAGGTGTCCAACAAGGCCCTGCCTGCTCCTATCG
AAAAGACCATCAGCAAGGCTAAGGGCCAGCCTCGGGAACCTCAGGTTTACACCCTGCCTCCATCTCGGGAAGAGATG
ACAAAGAACCAGGTGTCCCTGACCTGCCTGGTCAAGGGCTTCTACCCTTCCGACATTGCCGTGGAATGGGAGTCCAA
TGGCCAGCCTGAGAACAACTACAAGACAACCCCTCCTGTGCTGGACTCCGACGGCTCATTCTTCCTGTACTCCAAGC
TGACAGTGGACAAGTCTCGGTGGCAGCAGGGCAACGTGTTCTCCTGTTCTGTGATGCACGAGGCCCTGCACAACCAC
TACACCCAGAAGTCCCTGTCTCTGTCCCCTGGCAAGTGATGA
>PA015_LC (SEQ ID NO: 41)
ATGGGCTGGTCCTGCATCATTCTGTTTCTGGTGGCTACCGCCACCGGCGCTCATTCTGATGTGGTCATGACACAGAC
CCCTCTGAGCCTGTCTGTGACACCTGGACAGCCTGCCTCCATCTCCTGCAAGTCCTCTCAGTCCATCGTGCACAGCA
ACGCCCACACCTACCTGGAATGGTATCTGCAGAAGCCCGGCCAGTCTCCTCAGCTGCTGATCTACAAGGTGTCCAAC
AGATTCTCTGGCGTGCCCGACAGATTCAGCGGCTCTGGCTCTGGCACCGACTTCACCCTGAAGATCTCTAGAGTGGA
AGCCGAGGACGTGGGCGTGTACTACTGTTTCCAAGGCTCTCACGTGCCCGTGACCTTTGGCCAGGGAACAAAGCTGG
AAATCAAGCGGACCGTGGCCGCTCCTTCCGTGTTCATCTTTCCACCTTCCGACGAGCAGCTGAAGTCCGGCACAGCT
TCTGTCGTGTGCCTGCTGAACAACTTCTACCCTCGGGAAGCCAAGGTGCAGTGGAAAGTGGATAATGCCCTGCAGTC
CGGCAACTCCCAAGAGTCTGTGACCGAGCAGGACTCCAAGGACTCTACCTACTCTCTGTCCTCCACACTGACCCTGT
CCAAGGCCGACTACGAGAAGCACAAGGTGTACGCCTGTGAAGTGACCCACCAGGGACTGTCTAGCCCCGTGACCAAG
TCTTTCAACCGGGGCGAGTGCTGATGA

### EXAMPLES

### Example 1: Generation of Anti-KLRG1 Antibodies

The PA015 antibodies are humanized IgG1 antibodies against the extracellular domain of KLRG1. Mouse monoclonal antibodies (MAB) against human KLRG1 was generated by standard immunizations of female B ALB/c mice and SJL mice with human and cynomolgus KLRG1, and subsequent hybridoma screening. Several immunization strategies have been employed to generate a diverse number of antibody hits. Briefly SJL and Balb/c mice were repeatedly immunized with either cDNA, recombinant antigen or CHO cells expressing the antigen of interest. Antigen specific antibody titers were periodically monitored by ELISA and animals were sacrificed when appropriate titers were reached, usually between 1:1000 and 1:10000 dilution factor. Splenocytes from sacrificed mice were fused to mouse myeloma cells to produce hybridoma cells and later cultured and sub-cloned into single cells. Stable clones were scaled up and condition media was harvested and tested for expression of anti-KLRG1 antibodies by ELISA and FACS.

### Example 2: PA015 Binding to CHOK1 Cells Expressing Human KLRG1

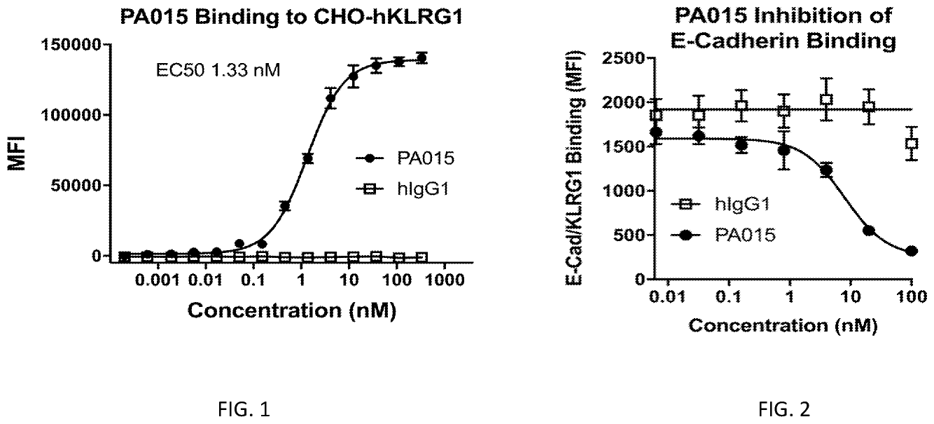

Binding of anti-KLRG1 monoclonal antibodies to cell expressed human and cynomolgus-KLRG1 is carried out by FACS. Chinese hamster ovary (CHO) cells are stably transfected to express full length human KLRG1 (CHOK1-hKLRG1)). CHOK1-hKLRG1 expressing cells were cultured and incubated with varying concentrations of either PA015 or control human IgG1. Cells were then centrifuged, the supernatant discarded, and washed with an incubation buffer (HBSS+2% FBS+10 mM Ca2+). Cells were resuspended and incubated with anti-human IgG-Alexa 488, washed, resuspended, and underwent FACS study. The mean fluorescent intensity (MFI) of the cells incubated with PA015 and with control human IgG1 are shown in FIG. 1. The EC50 for PA015 was 1.33 nM.

### Example 3: PA015 Inhibition of E-Cadherin Binding to CHOK1 Cells Expressing KLRG1 in Relative Binding Assay

The ability of monoclonal antibodies to inhibit E-cadherin/KLRG1 binding is measured by FACS.

CHOK1-hKLRG1 expressing cells were cultured and incubated with varying concentrations of either PA015 or control human IgG1. Recombinant human E-cadherin-ECD-FC-biotin at 0.3 μM was then added and incubated. Cells were then centrifuged, the supernatant discarded, and washed with an incubation buffer (HBSS+2% FBS+10 mM Ca2+). Cells were resuspended and incubated with Streptavidin-Alexa 488, washed, resuspended, and underwent FACS study. The mean fluorescent intensity (MFI) of the cells incubated with PA015 and with control human IgG1 are shown in FIG. 2. The IC50 value is determined by monitoring the loss of E-cadherin binding as a function of varying the concentration of monoclonal antibodies from 0.1 to 100 nM. The IC50 for PA015 inhibition of E-cadherin binding was 8.05 nM.

### Example 4: Superior Purity of PA015 Compared to HG1N02

Examples 4-6 refer to antibody PA015 described herein and an antibody designated HG1N02. HG1N02 is anti-KLRG1 antibody described in WO 2020/060781.

PA015 and HG1N02 were produced using the ExpiCho expression system (Thermo Fisher) at 1 L scale. Reducing capillary electrophoresis (rCE) and non-reducing capillary electrophoresis (nrCE) were performed, and the percentage of the main peak measured in 3 independent experiments. The results show superior purity during CHO cell production of PA015 compared to HG1N02 (Table 3).

TABLE 3
Purity of PA015 compared to HG1N02

| Purity | |
|---|---|
| rCE Main Peak | nrCE Main Peak |

| Trial | PA015 | HG1N02 | PA015 | HG1N02 |
|---|---|---|---|---|
| Trial 1 | 95.7% | 91.8% | 96.7% | 97.1% |
| Trial 2 | 96.7% | 95.0% | 98.4% | 92.8% |
| Trial 3 | 97.0% | 96.7% | 97.3% | 96.9% |
| Mean | 96.5% | 94.5% | 97.5% | 95.6% |

### Example 5: Superior Thermal Properties (Melting and Aggregation Temperatures) of PA015 Compared to HG1N02

PA015 and HG1N02 were produced using the ExpiCho expression system (Thermo Fisher) at 1 L scale. Melting temperature (Tm) was measured by full-spectrum fluorescence, and small molecule aggregate formation (Tagg 266) and large molecule aggregate formation (Tagg 473) were measured by static light scattering (SLS) using UNCLE (Unchained Labs, Inc.) in 3 independent experiments, at 4 hour, 7 day, and 28 day timepoints. The results show superior thermal properties (higher melting points and aggregation temperatures) for PA015 compared to HG1N02 (Table 4).

TABLE 4
Thermal properties of PA015 compared to HG1N02.
Temperatures shown in degrees Celsius

| Thermal | Tm1 | | Tagg 266 | | Tagg 473 | |
|---|---|---|---|---|---|---|
| Trial | PA015 | HG1N02 | PA015 | HG1N02 | PA015 | HG1N02 |
| Trial 1 | 69.3 | 68.3 | 73.8 | 70.9 | 75.0 | 71.9 |
| Trial 2 | 69.5 | 68.9 | 73.3 | 71.0 | 74.1 | 71.7 |
| Trial 3 | 69.3 | 68.7 | 74.0 | 71.6 | 74.8 | 72.4 |
| Mean | 69.4 | 68.6 | 73.7 | 71.2 | 74.6 | 72.0 |

### Example 6: Superior Thermal Properties (Melting and Aggregation Temperatures) After Stress Testing of PA015 Compared to HG1N02

PA015 and HG1N02 were subjected to stress testing with thermal stress, low pH, and high pH stress for 4 hours, for 7 days, and for 28 days. Melting temperature (Tm) was measured by full-spectrum fluorescence, and small molecule aggregate formation (Tagg 266) and large molecule aggregate formation (Tagg 473) were measured by static light scattering (SLS) using UNCLE (Unchained Labs, Inc.) at 4 hour, 7 day, and 28 day timepoints. The results show superior stability of thermal properties (higher melting points and aggregation temperatures) after stress testing for PA015 compared to HG1N02 (Tables 5-7).

TABLE 5
Thermal properties after 40° C. stress of PA015
and HG1N02. Temperatures shown in degrees Celsius

| 40° C. | Tm1 | | Tagg 266 | | Tagg 473 | |
|---|---|---|---|---|---|---|
| TimePoint | PA015 | HG1N02 | PA015 | HG1N02 | PA015 | HG1N02 |
| 4 Hours | 69.6 | 68.8 | 73.6 | 71.2 | 74.8 | 72.4 |
| Day 7 | 65.7 | 64.7 | 72.0 | 69.7 | 73.3 | 71.7 |
| Day 28 | 69.1 | 68.3 | 73.8 | 71.3 | 75.0 | 72.5 |

TABLE 6
Thermal properties after low pH (3.5) stress of PA015
and HG1N02 Temperatures shown in degrees Celsius.

| pH 3.5 | Tm1 | | Tagg 266 | | Tagg 473 | |
|---|---|---|---|---|---|---|
| TimePoint | PA015 | HG1N02 | PA015 | HG1N02 | PA015 | HG1N02 |
| 4 Hours | 69.0 | 67.8 | 73.4 | 70.5 | 74.5 | 71.8 |
| Day 7 | 68.9 | 68.1 | 72.8 | 69.8 | 74.3 | 71.9 |
| Day 28 | 68.4 | 68.8 | 73.1 | 70.3 | 74.5 | 71.1 |

TABLE 7
Thermal properties after high pH (8.5) stress of PA015
and HG1N02 Temperatures shown in degrees Celsius.

| pH 8.5 | Tm1 | | Tagg 266 | | Tagg 473 | |
|---|---|---|---|---|---|---|
| TimePoint | PA015 | HG1N02 | PA015 | HG1N02 | PA015 | HG1N02 |
| 4 Hours | 69.3 | 69.1 | 73.5 | 70.9 | 74.1 | 72.2 |
| Day 7 | 69.2 | 68.3 | 73.2 | 70.4 | 74.4 | 72.2 |
| Day 28 | 69.4 | 68.0 | 73.8 | 70.9 | 74.2 | 71.8 |

### Example 7: PA015 has Superior Blocking Compared with HG1N01 and HG1N02 of E-Cadherin to CHOK1 Cells Expressing KLRG1 in Relative Binding Assay

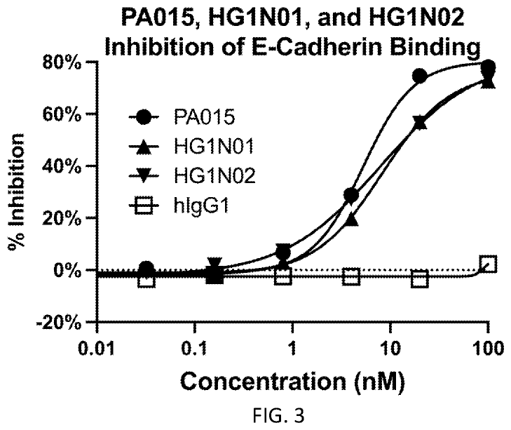

CHOK1-hKLRG1 expressing cells were cultured and incubated with varying concentrations of PA015, HG1N01, HG1N02 or control human IgG1. Recombinant human E-cadherin-ECD-FC-biotin at 0.3 μM was then added and incubated. Cells were centrifuged, the supernatant discarded, and washed with an incubation buffer (HBSS+2% FBS+10 mM Ca2+). Cells were resuspended and incubated with Streptavidin-Alexa 488, washed, resuspended, and underwent FACS study. The mean fluorescent intensity (MFI) of the cells incubated with the antibodies is shown in FIG. 3. The IC50 for PA015 was 5.3 nM, for HG1N01 8.3 nM, and for HG1N02 7.9 nM.

### Example 8: PA015 has Superior Binding to Human CD8+ T Cells Compared With HG1N02 in FACS Binding Assay

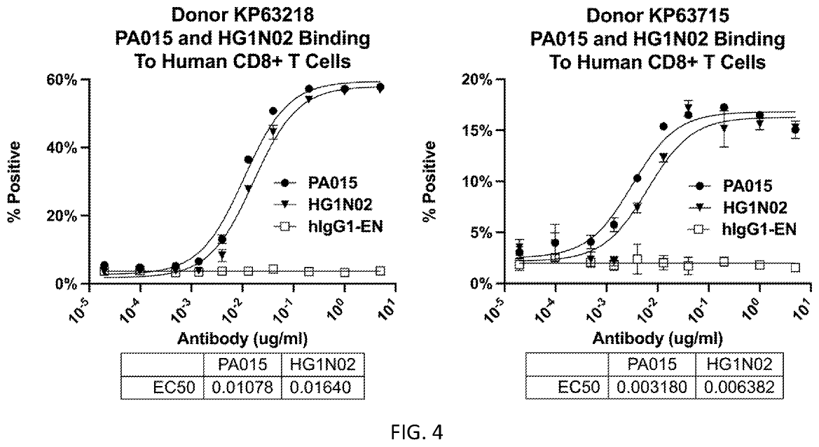

Human CD8+ T cells were isolated by magnetic separation from 3 healthy donors and incubated with varying concentrations of PA015, HG1N02, or control human IgG1 and detected with fluorescently labelled anti-human-IgG secondary antibodies. The percentage of positive cells and EC50 values are shown in FIG. 4.

### Example 9: PA015 has Superior Pharmacokinetic Properties Compared to HG1N01 in Non-Human Primates

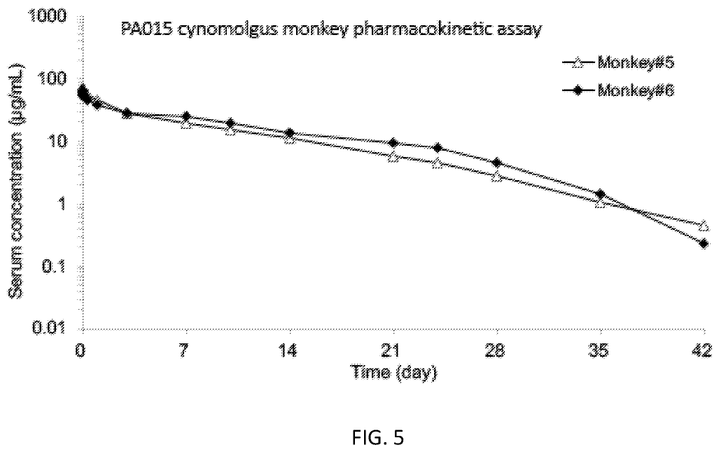

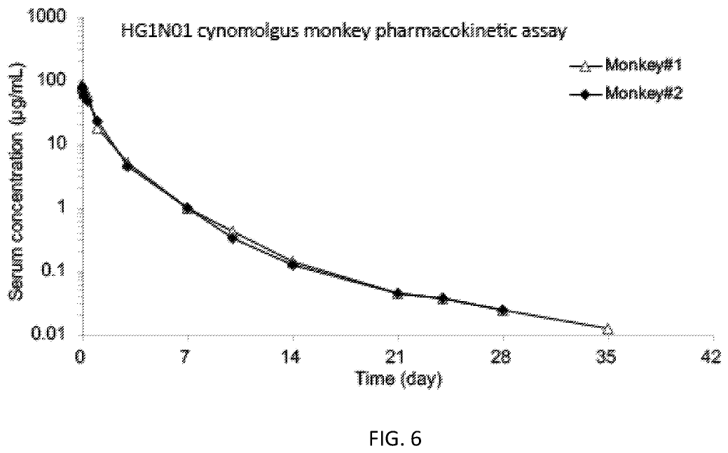

PA015 and HG1N01 (3 mg/kg) were administered as a single dose intravenously to two cynomolgus monkeys each. Serum concentration of each antibody was measured over 42 days (FIG. 5 and FIG. 6). PA015 in comparison to HG1N01 demonstrates superior clearance (6.99 vs 38.6 mL/day/kg), area under the curve (AUC; 429 vs 77.6 day*ug/mL) and beta half-life (6.23 vs 4.7 days) (Tables 8 and 9).

TABLE 8
Pharmacokinetic parameters of PA015

| PA015 PK | | | | |
|---|---|---|---|---|
| parameters | Unit | Monkey#5 | Monkey#6 | Mean |
| CL | mL/day/kg | 6.97 | 7.01 | 6.99 |
| Vss | mL/kg | 64.6 | 58.9 | 61.7 |
| V1 | mL/kg | 44.2 | 40.9 | 42.6 |
| Alpha t_{1/2} | day | 0.414 | 0.0173 | 0.216 |
| Beta t_{1/2} | day | 6.64 | 5.83 | 6.23 |
| AUC | day*μg/mL | 431 | 428 | 429 |
| MRT | day | 9.27 | 8.4 | 8.84 |

TABLE 9
Pharmacokinetic parameters of HG1N01

| HG1N01 PK | | | | |
|---|---|---|---|---|
| parameters | Unit | Monkey#1 | Monkey#2 | Mean |
| CL | mL/day/kg | 38.8 | 38.5 | 38.6 |
| Vss | mL/kg | 63.8 | 62.6 | 63.2 |
| V1 | mL/kg | 42 | 43.1 | 42.6 |
| Alpha t_{1/2} | day | 0.679 | 0.704 | 0.692 |
| Beta t_{1/2} | day | 4.82 | 4.58 | 4.7 |
| AUC | day*μg/mL | 77.4 | 77.9 | 77.6 |
| MRT | day | 1.65 | 1.63 | 1.64 |

### Example 10: PA015 has Improved Deamidation Properties Compared to HG1N02

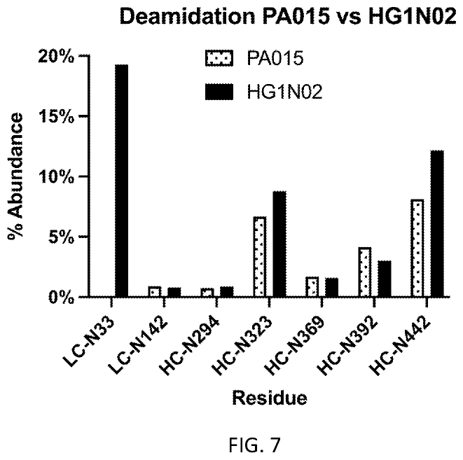

HG1N02 has a theoretical deamidation risk (NG at light chain residue N33). However, in a study of 131 clinically approved therapeutic mAbs (Lu Y et al. MAbs. 2019 January; 11(1):45-57), 48% (13/27) of CDR NG sites do not actually have deamidation under stress conditions, so sequence does not actually predict development of deamidation. Tryptic peptide mapping of unstressed PA015 and HG1N02 showed PA015 has less deamidation than HG1N02 at a key N33 light chain CDR residue (FIG. 7).

### INCORPORATION BY REFERENCE

The disclosure of each and every U.S. and foreign patent and pending patent application and publication referred to herein is specifically incorporated herein by reference in its entirety, as are the contents of Sequence Listing and Figures. The teachings of PCT/US2019/050110 (published as WO2020060781) are hereby incorporated by reference in their entirety.

### EQUIVALENTS

Those skilled in the art will understand that the disclosures described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure. Those skilled in the art will recognize or be able to ascertain using no more than routine experimentation, many equivalents of the specific embodiments described herein. Such equivalents are intended to be encompassed by the following claims. Any combination of the embodiments disclosed in the any plurality of the dependent claims or Examples is contemplated to be within the scope of the disclosure.

### OTHER EMBODIMENTS

From the foregoing description, it will be apparent that variations and modifications may be made to the invention described herein to adopt it to various usages and conditions, including the use of different signal sequences and nucleic acid sequences to express the antibodies of the invention having the same or similar protein sequence are considered to be within the scope of the invention. Other embodiments according to the invention are within the following claims.

Recitation of a listing of elements in any definition of a variable herein includes definitions of that variable as any single element or combination (or sub combination) of listed elements. Recitation of an embodiment herein includes that embodiment as any single embodiment or in combination with any other embodiments or portions thereof.

All publications and patent applications mentioned in the specification are indicative of the level of skill of those skilled in the art to which this disclosure pertains.

1. An antibody, or antigen-binding fragment thereof, that specifically binds to an extracellular domain of KLRG1 which comprises a heavy chain variable region comprising SEQ ID NO: 4.

2. The antibody, or antigen-binding fragment thereof, of claim 1, further comprising a light chain comprising a CDR-L1 selected from the group consisting of SEQ ID NO:13, SEQ ID NO:19, SEQ ID NO:25, SEQ ID NO:31, and SEQ ID NO:37; a CDR-L2 selected from the group consisting of SEQ ID NO:14, SEQ ID NO:20, SEQ ID NO:26, SEQ ID NO:32, and SEQ ID NO:38; and a CDR-L3 selected from the group consisting of SEQ ID NO: 15, SEQ ID NO:21, SEQ ID NO:27, SEQ ID NO:33, and SEQ ID NO:39.

3. The antibody, or antigen-binding fragment thereof, of claim 2, wherein the CDR-L1, CDR-L2, and CDR-L3 are SEQ ID NO: 13, SEQ ID NO: 14 and SEQ ID NO:15, respectively; SEQ ID NO:19, SEQ ID NO:20 and SEQ ID NO:21, respectively; SEQ ID NO:25, SEQ ID NO:26 and SEQ ID NO:27, respectively; SEQ ID NO:31, SEQ ID NO:32 and SEQ ID NO:33, respectively; or SEQ ID NO:37, SEQ ID NO:38 and SEQ ID NO:39, respectively.

4. An antibody, or antigen-binding fragment thereof, that specifically binds to an extracellular domain of KLRG1, which comprises a light chain variable region comprising SEQ ID NO: 5.

5. The antibody, or antigen-binding fragment thereof, of claim 4, further comprising a heavy chain comprising a CDR-H1 selected from the group consisting of SEQ ID NO:10, SEQ ID NO: 16, SEQ ID NO:22, SEQ ID NO:28, SEQ ID NO:34; a CDR-H2 selected from the group consisting of SEQ ID NO:11, SEQ ID NO:17, SEQ ID NO:23, SEQ ID NO:29, SEQ ID NO:35; and a CDR-H3 selected from the group consisting of SEQ ID NO: 12, SEQ ID NO: 18, SEQ ID NO:24, SEQ ID NO:30, SEQ ID NO:36.

6. The antibody, or antigen-binding fragment thereof, of claim 5, wherein the CDR-H1, CDR-H2, and CDR-H3 are SEQ ID NO: 10, SEQ ID NO:11 and SEQ ID NO:12, respectively; SEQ ID NO:16, SEQ ID NO:17 and SEQ ID NO:18, respectively; SEQ ID NO:22, SEQ ID NO:23 and SEQ ID NO:24, respectively; SEQ ID NO:28, SEQ ID NO:29 and SEQ ID NO:30, respectively; or SEQ ID NO:34, SEQ ID NO:35 and SEQ ID NO:36, respectively.

7. An antibody, or antigen-binding fragment thereof, that specifically binds to an extracellular domain of KLRG1which comprises:
(a) a heavy chain variable region comprising SEQ ID NO: 4, and
(b) a light chain variable region comprising SEQ ID NO: 5.

8. An antibody, or antigen-binding fragment thereof, that specifically binds to an extracellular domain of KLRG1, and which comprises a heavy chain comprising SEQ ID NO: 8.

9. An antibody, or antigen-binding fragment thereof, that specifically binds to an extracellular domain of KLRG1, and which comprises a light chain comprising SEQ ID NO: 9.

10. An antibody, or antigen-binding fragment thereof, that specifically binds to an extracellular domain of KLRG1, and which comprises:
(a) a heavy chain comprising SEQ ID NO: 8, and
(b) a light chain comprising SEQ ID NO: 9.

11. The antibody, or antigen-binding fragment of claim 1, wherein said antigen-binding fragment is a F(ab) fragment.

12. The antibody, or antigen-binding fragment of claim 1, wherein said antigen-binding fragment is a F(ab₂) fragment.

13. The antibody, or antigen-binding fragment of claim 1, wherein said antibody or antigen-binding fragment thereof is bispecific.

14. A method of treating cancer, comprising administering to a cancer patient the antibody or antigen-binding fragment thereof of claim 1.

15. The method of claim 14, further comprising administering a checkpoint inhibitor to said cancer patient.

16. The method of claim 15, wherein said administering of the checkpoint inhibitor is done at the same time as administering the antibody or antigen-binding fragment thereof.

17. The method of claim 15, wherein the checkpoint inhibitor is administered within one day, one week, or one month of the antibody or antigen-binding fragment thereof.

18. The method of claim 14, wherein said cancer is one or more of breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma

19. An adjunct therapy, comprising administering to a subject who is undergoing treatment with a checkpoint inhibitor the antibody or antigen-binding fragment thereof of claim 1, thereby to perform said adjunct therapy.

20. An isolated nucleic acid encoding the antibody or antigen binding fragment thereof, of claim 1.

21. The isolated nucleic acid of claim 20, comprising SEQ ID NO:40.

22. The isolated nucleic acid of claim 20, comprising SEQ ID NO:41.

23. An expression vector comprising the nucleic acid of claim 20.

24. A host cell comprising the expression vector of claim 23.
